SEQUENCE LISTING (6 sequences)

SEQ ID NO: 1 - protein, 35 aa, artificial
  synthetic
  DOMAIN (1)..(35): Module C (ADF-4)
GSSAAAAAAAASGPGGYGPENQGPSGPGGYGPGGP

SEQ ID NO: 2 - protein, 35 aa, artificial
  synthetic
  DOMAIN (1)..(35): Module Cc
GSSAAAAAAAASGPGGYGPENQGPCGPGGYGPGGP

SEQ ID NO: 3 - protein, 560 aa, artificial
  synthetic
  REPEAT (1)..(560): C16
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP

SEQ ID NO: 4 - protein, 1120 aa, artificial
  synthetic
  REPEAT (1)..(1120): C32
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP

SEQ ID NO: 5 - protein, 1680 aa, artificial
  synthetic
  REPEAT (1)..(1680): C48
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP

SEQ ID NO: 6 - protein, 280 aa, artificial
  synthetic
  REPEAT (1)..(280): C8
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP

(19) United States
(12) Patent Application Publication
(10) Pub. No.: US 2020/0270316 A1
(43) Pub. Date: Aug. 27, 2020

(54) Silk Alcohol Formulations

(71) Applicant: GIVAUDAN SA, Vernier (CH)

(72) Inventors: Lin RÖMER, Ottobrunn (DE); Jens KLEIN, Pullachi (DE); Ralf MEHRWALD, München (DE)

(21) Appl. No.: 16/762,058

(22) PCT Filed: Nov. 8, 2018
(86) PCT No.: PCT/EP2018/080557

§ 371 Date: May 6, 2020

(30) Foreign Application Priority Data
Nov. 10, 2017 (EP) 17201049.8

(51) Int. Cl. C07K 14/435; A61K 8/64; A61Q 13/00; A61K 31/16; A61K 9/06

(52) U.S. Cl. CPC C07K 14/43536; A61K 8/64; A61K 38/00; A61K 31/16; A61K 9/06; A61Q 13/00

(57) ABSTRACT

The present invention relates to an aqueous formulation comprising a structural protein and an alcohol. Further, the present invention relates to a method for producing an aqueous formulation. Furthermore, the present invention relates to a pharmaceutical composition comprising the aqueous formulation comprising a structural protein and an alcohol. In addition, the present invention relates to a cosmetic composition comprising the aqueous formulation comprising a structural protein and an alcohol.

The present invention relates to an aqueous formulation comprising a structural protein and an alcohol. Further, the present invention relates to a method for producing an aqueous formulation. Furthermore, the present invention relates to a pharmaceutical composition comprising the aqueous formulation comprising a structural protein and an alcohol. In addition, the present invention relates to a cosmetic composition comprising the aqueous formulation comprising a structural protein and an alcohol.

### BACKGROUND OF THE INVENTION

The use of natural structural proteins such as silk proteins is well known and has been widely practiced in the cosmetics field, in particular the use of silk proteins from spider or the silk worm Bombyx mori. Cosmetic formulations comprising silk provide, for example, moisture management and skin protection. In particular, silk acts as a natural humectant to hydrate and condition the skin leaving skin feeling softer and smoother. Silk forms a natural layer over the skin, keeping the moisture locked in and harsh conditions out, leaving skin protected and well-nourished. In hair care formulations, it further helps to make hair more smooth and nourished as well given it a lasting shine.

Because of its good tolerability, a structural protein formulation, e.g. a silk protein formulation, can also be used as a basic formulation to formulate, for example, pharmaceutical or cosmetic compounds, in order to produce pharmaceutical or cosmetic compositions. The formulation of poorly water soluble compounds such as oils with an aqueous structural protein solution, e.g. aqueous silk protein solution, is, however, generally not possible. In contrast thereto, poorly water soluble compounds can be mixed with solutions comprising alcohol. A structural protein, e.g. a silk protein, is, however, generally not soluble in solutions comprising alcohol.

Thus, there is a need for an effective and inexpensive process for the production of formulations comprising a structural protein such as a silk protein as a base material and water soluble, poorly water soluble as well as water insoluble compounds as additives. Said formulations may be used in the pharmaceutical and cosmetic field.

The present inventors were surprisingly be able to provide a production process for the generation of formulations comprising a structural protein such as a silk protein and alcohol. Said formulations can be used for the formulation of water soluble, poorly water soluble, and water insoluble compounds. The present inventors were further be able to provide a formulation comprising a structural protein such as a silk protein as well as an alcohol.

### SUMMARY OF THE INVENTION

In a first aspect, the present invention relates to an aqueous formulation comprising a structural protein and an alcohol.

In a second aspect, the present invention relates to a method for producing an aqueous formulation comprising a structural protein and an alcohol comprising the steps of:
(i) providing an aqueous solution comprising a structural protein and an aqueous solution comprising an alcohol, and
(ii) mixing the aqueous solutions, thereby obtaining an aqueous formulation comprising a structural protein and an alcohol.

In a third aspect, the present invention relates to an aqueous formulation comprising a structural protein and an alcohol obtainable by the method of the second aspect.

In a fourth aspect, the present invention relates to a method for producing an article comprising the steps of:
(i) providing an aqueous formulation comprising a structural protein and an alcohol according to the first or third aspect, and
(ii) forming an article out of/from the formulation provided in (i).

In a fifth aspect, the present invention relates to an article obtainable by the method of the fourth aspect.

In a sixth aspect, the present invention relates to a pharmaceutical composition comprising

the aqueous formulation comprising a structural protein and an alcohol according to the first or third aspect, or

the article according to the fifth aspect.

In a seventh aspect, the present invention relates to a cosmetic composition comprising the aqueous formulation comprising a structural protein and an alcohol according to the first or third aspect, or

the article according to the fifth aspect.

In an eight aspect, the present invention relates to

an aqueous formulation comprising a structural protein and an alcohol according to the first or third aspect, or

an article according the fifth aspect.

for use as a pharmaceutical.

In a ninth aspect, the present invention relates to the use of

the aqueous formulation comprising a structural protein and an alcohol according to the first or third aspect, or

the article according to the fifth aspect

for the protection of a compound.

In a tenth aspect, the present invention relates to the use of

the aqueous formulation comprising a structural protein and an alcohol according to the first or third aspect, or

the article according to the fifth aspect

for sustained or controlled release of a compound.

In an eleventh aspect, the present invention relates to the use of

the aqueous formulation comprising a structural protein and an alcohol according the first or third aspect, or

the article according to the fifth aspect

for prolongation of the retention time of a compound.

In a twelfth aspect, the present invention relates to the use of

the aqueous formulation comprising a structural protein and an alcohol according to the first or third aspect, or

the article according to the fifth aspect

for the formulation of a poorly water soluble, a water insoluble, a lipophilic, or an oily compound.

### DETAILED DESCRIPTION OF THE INVENTION

### DEFINITIONS

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention. In the event of a conflict between the definitions or teachings of such incorporated references and definitions or teachings recited in the present specification, the text of the present specification takes precedence.

The term “comprise” or variations such as “comprises” or “comprising” according to the present invention means the inclusion of a stated integer or group of integers but not the exclusion of any other integer or group of integers. The term “consisting essentially of” according to the present invention means the inclusion of a stated integer or group of integers, while excluding modifications or other integers which would materially affect or alter the stated integer. The term “consisting of” or variations such as “consists of” according to the present invention means the inclusion of a stated integer or group of integers and the exclusion of any other integer or group of integers.

The terms “a” and “an” and “the” and similar reference used in the context of describing the invention (especially in the context of the claims) are to be construed to cover both the singular and the plural, unless otherwise indicated herein or clearly contradicted by context.

The term “aqueous formulation”, as used herein, refers to a formulation having a clear appearance. It does not comprise visible aggregates and/or precipitates. Said visible aggregates and/or precipitates are usually the cause for clouding. The term “aqueous formulation”, as used herein, also refers to a homogenous formulation comprising fibrillary complexes of structural proteins, wherein the structural proteins are homogenously distributed in the aqueous formulation. In said fibrillary complexes, the structural proteins are oriented and/or conjoined to each other. Said fibrillary complexes of structural proteins may be formed by self-assembling of the structural proteins in the aqueous formulation. Said mechanism of self-assembling may include covalent and/or non-covalent interactions between the structural proteins.

In a preferred embodiment, the aqueous formulation is an aqueous gel, in particular a hydrogel. In a more preferred embodiment, the aqueous formulation is a flowable or a non-flowable hydrogel. In another more preferred embodiment, the aqueous formulation is an aqueous dispersion. In another more preferred embodiment, the aqueous dispersion is in a liquid, viscous, gel-like, or solid state. The presence of a clear appearance can be determined by measurement of the optical density.

In contrast thereto, a turbid aqueous formulation comprises visible aggregates and/or precipitates. The structural proteins comprised therein show a diffuse and unoriented aggregation. They have mainly a random orientation and are not fibrillary.

The term “flowable hydrogel”, as used herein, refers to a hydrogel that is able/capable of flowing or being flowed. In a preferred embodiment, the hydrogel is in a liquid state.

The term “non-flowable hydrogel”, as used herein, refers to a formulation that is non able/capable of flowing or being flowed. In a preferred embodiment, the hydrogel is in a solid state.

The followability of a hydrogel can easily be determined by the skilled person, e.g. by rheology or viscosity measurements. The followability measurements are preferably preformed under standard conditions (25° C.)

Due to the biocompatibility, biodegradability and low immunogenicity, structural proteins have a high potential for a variety of applications when processed into morphologies such as films, coatings, fibers, porous structures such as scaffolds or foams, particles, capsules, or gels like hydrogels. For example, if the concentration of the structural protein is below a certain threshold, “particles” can be formed by nucleation and growth. A “fiber” can be obtained by spinning a fiber out of an aqueous solution (spinning solution). A “film” can be obtained by simple evaporation of the solvent. If porogens are introduced into the structural protein solution and the solution is subsequently evaporated, porous structures such as “scaffolds” or “foams” can be produced.

The term “hydrogel”, as used herein, refers to a structure that is formed if the concentration of structural proteins is high enough to build a continuous network by which the liquid component is immobilized. Said network is preferably formed by self-assembling of the structural proteins providing the basis of the silk hydrogel. In particular, the hydrogel is a hydrophilic polymeric network of structural proteins. Said network is stabilized by chemical and/or physical interactions between the structural proteins. The network is dispersed throughout an immobilized aqueous phase. The hydrophilicity and stability of the hydrogel permits the penetration and absorption of water (swelling) without dissolving, thus, maintaining its three-dimensional (3D) structure and function. The hydrogel is an excellent material candidate for a variety of biomedical, biological, pharmaceutical, or cosmetic applications. These applications include, but are not limited to, drug and cosmetic compound delivery vehicles.

The term “structural protein”, as used herein, refers to any protein which comprises repeat units/repeating building blocks made of amino acids. The structural protein has preferably the ability to self-assemble. In particular, the structural protein is capable of forming fibrillary protein complexes in the aqueous formulation, e.g. hydrogel. The structural protein may be selected from the group consisting of a silk protein, keratin, collagen, and elastin. The structural protein is preferably a recombinant protein. It is particularly preferred that the structural protein is a silk protein such as a spider silk protein. An exemplarily process for producing a silk protein which may be used in the present invention is described in WO 2006/008163 and in WO 2011/120690.

The terms “protein” and “polypeptide” are used interchangeably in the context of the present invention. They refer to a long peptide-linked chain of amino acids, e.g. one that is at least 40 amino acids long.

The term “silk protein”, as used herein, refers to a protein which shows, in comparison to other proteins, a quite aberrant amino acid composition. In particular, a silk protein possess large quantities of hydrophobic amino acids such as glycine or alanine. In addition, a silk protein contains highly repetitive amino acid sequences or repetitive units (repeat units, modules), especially in their large core domain.

Based on DNA analysis, it was shown that all silk proteins are chains of repetitive units which further comprise a limited set of distinct shorter peptide motifs. The expressions “peptide motif” and “consensus sequence” can be used interchangeably herein. Generally, the silk consensus sequences can be grouped into four major categories: GPGXX, GGX, Aₓ or (GA)ₙ and spacers. These categories of peptide motifs in silk proteins have been assigned structural roles. For example, it has been suggested that the GPGXX motif is involved in a β-turn spiral, probably providing elasticity. The GGX motif is known to be responsible for a glycine-rich 3₁-helix. Both GPGXX and GGX motifs are thought to be involved in the formation of an amorphous matrix that connects crystalline regions, thereby providing elasticity of the fiber. Alanine-rich motifs typically contain 6-9 residues and have been found to form crystalline β-sheets. The spacers typically contain charged groups and separate the iterated peptide motifs into clusters. The silk protein can perform self-assembly. Preferably, the silk protein is a spider silk protein. More preferably, the silk polypeptide, e.g. spider silk protein, is a recombinant protein.

The term “self-assembly”, as used herein, refers to a process in which a disordered system of pre-existing proteins forms an organized structure or pattern as a consequence of specific, local interactions (e.g. van der Waals forces, hydrophobic interactions, hydrogen bonds, and/or salt-bridges, etc.) among the proteins themselves, without external direction or trigger although external factors might influence speed and nature of self-assembly. This particularly means that when two or more disordered and/or unfolded proteins are brought into contact, they interact with each other and consequently form a three dimensional structure. The change from a disordered system to an organised structure or pattern during self-assembly is characterized by a transition from a fluid state to a gelatinous/gel-like and/or solid state and a corresponding increase in viscosity. The transition from a fluid state to a gelatinous/gel-like state can be monitored, for example, by optical measurement or rheology. These techniques are known to the skilled person. The transition from a fluid state to a solid state can be monitored, for example, using optical methods.

The term “article”, as used herein, refers to any object that may be produced out off/from the aqueous formulation. The article may be selected from the group consisting of a gel such as a hydrogel, a film, a particle, a capsule, a fiber, and a porous structure such as a scaffold or a foam.

The term “compound”, as used herein, refers to any compound having a purpose that may be useful in the present invention, e.g. a compound that can be delivered to a subject/patient. The compound may be selected from the group consisting of a pharmaceutical compound such as a drug, a cosmetic compound such as a fragrance, a flavour, a chemical compound, a detergent compound, a coloring compound such as a dye, a nutrient, or a dietary supplement.

The term “pharmaceutical compound”, as used herein, refers to any biological or chemical substance, particularly pharmacological, metabolic, or immunological substance, which may be used in the treatment, cure, prophylaxis, prevention, or diagnosis of a pathological condition, e.g. a disease or disorder, or which may be used to otherwise enhance physical, psychical or mental well-being. Accordingly, the term “pharmaceutical compound” envisaged in the context of the present invention includes any compound with therapeutic, diagnostic, or prophylactic effects. For example, the pharmaceutical compound can be a compound that affects or participates in tissue growth, cell growth, cell differentiation, a compound that is able to invoke a biological action such as an immune response, or a compound that can play any other role in one or more biological processes. Preferably, the pharmaceutical compound is selected from the group consisting of an anti-microbial compound, such as an antibacterial compound (e.g. an antibiotic), an anti-viral compound or an anti-fungal compound, an immunosuppressive compound, an anti-inflammatory compound, an anti-allergic compound, an anti-coagulant, an anti-rheumatic compound, an anti-psoriatic compound, a sedative compound, a muscle relaxant, an anti-migraine compound, an anti-depressant, an insect repellent, a growth factor, a hormone, a hormone antagonist, an antioxidant, a protein, such as a glycoprotein, lipoprotein, or an enzyme (e.g. hyaluronidases), a polysaccharide, a free radical scavenger, a radio-therapeutic compound, a photodynamic therapy compound, a dye such as a fluorescent dye, and a contrast agent.

The term “cosmetic compound” as used herein, refers to a substance intended mainly for external use on the body surface, e.g. human body surface, or in the oral cavity, e.g. of a human, for cleaning and personal hygiene to alter the appearance or body odor or to convey scent. In particular, it is meant that a cosmetic substance is a molecule which shows a certain predictable effect. Such an effect molecule can be, for example, a proteinaceous molecule (e.g. an enzyme) or a non-proteinaceous molecule (e.g. a fragrance, flavor, dye, pigment, photo-protective agent, vitamin, provitamin, an antioxidant, conditioner, or a compound comprising metal ions). The term “cosmetic compound” also refers to cleansing substances.

The term “detergent compound”, as used herein, refers to any detergent substance or washing active substance. Such detergent substance can be for example a cleaning agent or a laundry detergent.

The compound may be water soluble, poorly water soluble or water insoluble.

The term “water-soluble compound”, as used herein, refers to any ionic compound (or salt) which is able to dissolve in water. Generally, the underlying solvation arises because of the attraction between positive and negative charges of the compound with the partially-negative and partially positive charges of the H₂O-molecules, respectively. Substances or compounds which dissolve in water are also termed “hydrophilic” (“water-loving”). Water solubility, also known as aqueous solubility, is the maximum amount of a substance that can dissolve in water at equilibrium at a given temperature and pressure. Generally, the limited amount is given by the solubility product.

In the context of the present invention “water-soluble” means a water solubility of 10 g compound or more per 1 liter of water at 20° C. Preferably, the water solubility is at least 20 g, at least 30 g, at least 40 g, and at least 50 g compound per 1 liter of water, more preferably at least 60 g, at least 70 g, at least 80 g, at least 90 and at least 100 g compound per 1 liter of water, and most preferably at least 200 g, at least 300 g, at least 400 g, at least 500 g, and at least 800 g compound per 1 liter of water. Compounds which are water soluble typically comprise the following chemical groups: cationic groups such as metallic cations, ammonium cations and/or anionic groups such as acetate, nitrate, chloride, bromide, iodide or sulphate.

The term “poorly water soluble”, as used herein, refers to a water solubility of less than 10 g compound per 1 liter of water at 20° C. In particular, poorly water soluble refers to a water solubility of less than 10 compound per 1 liter of water and more than 5 g compound per liter of water at 20° C.

The term “water insoluble”, as used herein, refers to a water solubility of less than 5 g compound per 1 liter of water at 20° C., preferably less than 1 g compound per 1 liter of water at 20° C., more preferably less than 0.5 g compound per 1 liter of water at 20° C., even more preferably less than 0.1 g compound per 1 liter of water at 20° C.

Typical measures for water solubility used in organic chemistry and the pharmaceutical sciences are a partition (P) or distribution coefficient (D), which give the ratio of concentrations of a compound in the two phases of a mixture of two immiscible solvents at equilibrium. Methods for determining the log P value of a compound are for example the shake flask (or tube) method, HPLC or electrochemical methods such as ITIES (Interfaces between two immiscible electrolyte solutions). Preferably, the log P value can be predicted using ACDlogP-Software (available at Advanced Chemistry Development, ACD/labs).

The pharmaceutical composition of the present invention may further comprise pharmaceutical acceptable carriers, diluents, and/or excipients.

The term “excipient”, as used herein, is intended to indicate all substances in a pharmaceutical composition which are not active ingredients such as binders, lubricants, thickeners, surface active agents, preservatives, emulsifiers, buffers, flavoring agents, or colorants.

The term “diluent”, as used herein, relates to a diluting and/or thinning agent. Moreover, the term “diluent” includes a solution, suspension (e.g. liquid or solid suspension) and/or media.

The term “carrier”, as used herein, relates to one or more compatible solid or liquid fillers, which are suitable for an administration, e.g. to a human. The term “carrier” relates to a natural or synthetic organic or inorganic component which is combined with an active component in order to facilitate the application of the active component. Preferably, carrier components are sterile liquids such as water or oils, including those which are derived from mineral oil, animals, or plants, such as peanut oil, soy bean oil, sesame oil, sunflower oil, etc. Salt solutions and aqueous dextrose and glycerin solutions may also be used as aqueous carrier compounds.

Pharmaceutically acceptable carriers or diluents for therapeutic use are well known in the pharmaceutical art, and are described, for example, in Remington's Pharmaceutical Sciences, Mack Publishing Co. (A. R Gennaro edit. 1985). Examples of suitable carriers include, for example, magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Examples of suitable diluents include ethanol, glycerol, and water.

Pharmaceutical carriers, diluents, and/or excipients can be selected with regard to the intended route of administration and standard pharmaceutical practice. The pharmaceutical compositions of the present invention may comprise as, or in addition to, the carrier(s), excipient(s) or diluent(s) any suitable binder(s), lubricant(s), suspending agent(s), coating agent(s), and/or solubilising agent(s). Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose, and polyethylene glycol. Examples of suitable lubricants include sodium oleate, sodium stearate, magnesium stearate, sodium benzoate, sodium acetate, sodium chloride, and the like. Preservatives, stabilizers, dyes, and even flavoring agents may be provided in the pharmaceutical composition. Examples of preservatives include sodium benzoate, sorbic acid, and esters of p-hydroxybenzoic acid. Antioxidants and suspending agents may be also used.

The terms “individual” and “subject” are used interchangeably in the context of the present invention. The individual or subject may be healthy, afflicted with a disease or disorder (e.g. cancer), or susceptible to a disease or disorder (e.g. cancer). The individual or subject may be an animal or a human. Preferably, the animal is a mammal (e.g. mouse, rat, rabbit, dog, cat, cattle, swine, sheep, horse or primate). Unless otherwise stated, the terms “individual” and “subject” do not denote a particular age and, thus, encompass adults, elderlies, children, and newborns. The “individual” or “subject” may be a “patient”.

The term “patient”, as used herein, means an individual or subject which is diseased, i.e. which suffers from a disease or disorder. The patient may be an animal, e.g. a human. Preferably, the animal is a human or another mammal (e.g. mouse, rat, rabbit, dog, cat, cattle, swine, sheep, horse or primate).

### EMBODIMENTS OF THE INVENTION

The present inventors were surprisingly be able to provide a production process for the generation of formulations comprising a structural protein such as a silk protein and alcohol. Said formulations can be used for the formulation of water soluble, poorly water soluble, and water insoluble compounds. The present inventors were further be able to provide a formulation comprising a structural protein such as a silk protein as well as an alcohol.

Thus, in a first aspect, the present invention relates to an aqueous formulation comprising a structural protein and an alcohol. In particular, the formulation has a clear appearance. It does not comprise visible aggregates and/or precipitates. Said visible aggregates and/or precipitates are usually the course for clouding. In addition, the formulation comprises fibrillary complexes of structural proteins. In said fibrillary complexes, the structural proteins are oriented and/or conjoined to each other. Said fibrillary complexes of structural proteins may be formed by self-assembling of the structural proteins in the aqueous formulation. Said mechanism of self-assembling may include covalent and/or non-covalent interactions between the structural proteins. The aqueous formulation may also be designated as aqueous dispersion. The presence of a clear appearance can be determined by measurement of the optical density.

In contrast thereto, a turbid aqueous formulation comprises visible aggregates and/or precipitates. The structural proteins comprised therein show a diffuse and unoriented aggregation. They have mainly a random orientation and are not fibrillary. The turbid aqueous formulation is usually a suspension.

In the aqueous formulation, the structural protein is preferably present in a concentration of between 0.05 wt % and 5 wt %, in particular of between 0.1 wt % and 5 wt %, between 0.2 wt % and 5 wt %, between 0.3 wt % and 5 wt %, between 0.4 wt % and 5 wt %, between 0.5 wt % and 5 wt %, between 0.6 wt % and 5 wt %, between 0.7 wt % and 5 wt %, between 0.8 wt % and 5 wt %, between 0.9 wt % and 5 wt %, between 1 wt % and 5 wt %, between 1.5 wt % and 4.5 wt %, between 2 wt % and 4 wt %, or between 2.5 wt % and 3.5 wt %, e.g. 0.05, 0.1, 0.2, 0.3, 0.4, 0.5, 0.6, 0.7, 0.8, 0.9, 1, 1.1, 1.175, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 wt %.

It is preferred that said formulation comprises

between 60 wt % and 90 wt % alcohol, in particular between 61 wt % and 89 wt %, between 62 wt % and 88 wt %, between 63 wt % and 87 wt %, between 64 wt % and 86 wt %, between 65 wt % and 85 wt %, between 66 wt % and 84 wt %, between 67 wt % and 83 wt %, between 68 wt % and 82 wt % alcohol, between 69 wt % and 81 wt %, between 70 wt % and 80 wt %, between 71 wt % and 79 wt %, between 72 wt % and 78 wt %, between 73 wt % and 77 wt %, or between 74 wt % and 76 wt % alcohol, e.g. 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 wt % alcohol,

between 0.05 wt % and 5 wt % of a structural protein, in particular between 0.1 wt % and 5 wt %, between 0.2 wt % and 5 wt %, between 0.3 wt % and 5 wt %, between 0.4 wt % and 5 wt %, between 0.5 wt % and 5 wt %, between 0.6 wt % and 5 wt %, between 0.7 wt % and 5 wt %, between 0.8 wt % and 5 wt %, between 0.9 wt % and 5 wt %, between 1 wt % and 5 wt %, between 1.5 wt % and 4.5 wt %, between 2 wt % and 4 wt %, or between 2.5 wt % and 3.5 wt %, e.g. 0.05, 0.1, 0.2, 0.3, 0.4, 0.5, 0.6, 0.7, 0.8, 0.9, 1, 1.5, 2, 2.5, 3, 3.5, 4, 4.5, or 5 wt % of a structural protein, and

between 5 wt % and 39.95 wt % water, in particular between 5 wt % and 39.9 wt %, between 5 wt % and 39.8 wt %, between 5 wt % and 39.7 wt %, between 5 wt % and 39.6 wt %, between 5 wt % and 39.5 wt %, between 5 wt % and 39.4 wt %, between 5 wt % and 39.3 wt %, between 5 wt % and 39.2 wt %, between 5 wt % and 39.1 wt %, between 5 wt % and 39 wt %, between 6 wt % and 38 wt %, between 7 wt % and 37 wt %, between 8 wt % and 36 wt %, between 9 wt % and 35 wt %, between 10 wt % and 34 wt %, between 11 wt % and 33 wt %, between 12 wt % and 32 wt %, between 13 wt % and 31 wt %, between 14 wt % and 30 wt %, between 15 wt % and 29 wt %, between 16 wt % and 28 wt %, between 17 wt % and 27 wt %, between 18 wt % and 26 wt %, between 19 wt % and 25 wt %, between 20 wt % and 24 wt %, or between 21 wt % and 23 wt %, e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 35.5, 36, 36.5, 37, 37.5, 38, 38.5, 39, 39.1, 39.2, 39.3, 39.4, 39.5, 39.6, 39.7, 39.8, 39.9, 39.95 wt % water.

It is more preferred that said formulation comprises

between 60 wt % and 80 wt % alcohol,

between 0.75 wt % and 2 wt % of a structural protein, and

between 18 wt % and 39.25 wt % water.

It is most preferred that the formulation comprises

70 wt % alcohol,

1.25 wt % of a structural protein and

28.75 wt % water.

The structural protein may be the silk protein C₈, C₁₆, C₃₂, or C₄₈.

The alcohol may be selected from the group consisting of ethanol, methanol, and isopropanol.

The ethanol may be ethanol having a purity of ≥99.5% (p.a.).

Preferably, the structural protein has a molecular weight of between 20 kDa and 140 kDa, more preferably of between 20 kDa and 95 kDa or between 30 kDa and 75 kDa, and even more preferably of between 40 kDa and 55 kDa. For example, the structural protein has a molecular weight of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, or 140 kDa.

The aqueous formulation may have a complex viscosity of between 0.04 Pa·s and 30 Pa·s, preferably of between 0.2 Pa·s and 30 Pa·s, and more preferably of between 0.8 Pa·s and 15 Pa·s.

The aqueous formulation has preferably a pH of >6.5, more preferably a pH of >7.0, and even more preferably of >8.0, e.g. a pH of >6.5, 7, 7.5, 8, 8.5, 9, 9.5, 10, 10.5, 11, 11.5, or 12.

In one embodiment, the aqueous formulation is a hydrogel. In particular, the hydrogel has a clear appearance. It does not comprise visible aggregates and/or precipitates. Said visible aggregates and/or precipitates are usually the cause for clouding. In addition, the hydrogel comprises fibrillary complexes of structural proteins. In said fibrillary complexes, the structural proteins are oriented and/or conjoined to each other. Said fibrillary complexes of structural proteins may be formed by self-assembling of the structural proteins. Said mechanism of self-assembling may include covalent and/or non-covalent interactions between the structural proteins.

The hydrogel may be a flowable hydrogel or a non-flowable hydrogel. The flowable hydrogel may also be designated as an aqueous dispersion in a liquid state. The non-flowable hydrogel may also be designated as an aqueous dispersion in a solid state.

In contrast thereto, a turbid hydrogel comprises visible aggregates and/or precipitates. The structural proteins comprised therein show a diffuse and unoriented aggregation. They have mainly a random orientation and are not fibrillary.

In the hydrogel, the structural protein is preferably present in a concentration of between 0.05 wt % and 5 wt %, in particular of between 0.1 wt % and 5 wt %, between 0.2 wt % and 5 wt %, between 0.3 wt % and 5 wt %, between 0.4 wt % and 5 wt %, between 0.5 wt % and 5 wt %, between 0.6 wt % and 5 wt %, between 0.7 wt % and 5 wt %, between 0.8 wt % and 5 wt %, between 0.9 wt % and 5 wt %, between 1 wt % and 5 wt %, between 1.5 wt % and 4.5 wt %, between 2 wt % and 4 wt %, or between 2.5 wt % and 3.5 wt %, e.g. 0.05, 0.1, 0.2, 0.3, 0.4, 0.5, 0.6, 0.7, 0.8, 0.9, 1, 1.1, 1.175, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 wt %.

The structural protein may be the silk protein C₈, C₁₆, C₃₂, C₄₈, or variants thereof.

In one preferred embodiment, the aqueous formulation is a flowable hydrogel. The flowable hydrogel may also be designated as an aqueous dispersion in a fluid state.

In the flowable hydrogel, the structural protein is preferably present in a concentration of between 0.05 wt % and 1.25 wt %, more preferably present in a concentration of between 0.75 wt % and 1.25 wt %, e.g. in a concentration of 0.05, 0.1, 0.2, 0.25, 0.3, 0.4, 0.5, 0.6, 0.7, 0.75, 0.8, 0.9, 1, 1.1, 1.2, or 1.25 wt %, wherein the structural protein is the silk protein C₁₆ or are variants thereof.

In one more preferred embodiment, the flowable hydrogel comprises

between 50 wt % and 90 wt % alcohol, in particular between 51 wt % and 89 wt %, between 52 wt % and 88 wt %, between 53 wt % and 87 wt %, between 54 wt % and 86 wt %, between 55 wt % and 85 wt %, between 56 wt % and 84 wt %, between 57 wt % and 83 wt %, between 58 wt % and 82 wt % alcohol, between 59 wt % and 81 wt %, between 60 wt % and 80 wt %, between 61 wt % and 79 wt %, between 62 wt % and 78 wt %, between 63 wt % and 77 wt %, between 64 wt % and 76 wt %, between 65 wt % and 75 wt %, between 66 wt % and 74 wt %, between 67 wt % and 73 wt %, between 68 wt % and 72 wt %, or between 69 wt % and 71 wt % alcohol, e.g. 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 wt % alcohol, between 0.05 wt % and 1.25 wt % of a structural protein, in particular between 0.1 wt % and 1.25 wt %, between 0.2 wt % and 1.25 wt %, between 0.3 wt % and 1.25 wt %, between 0.4 wt % and 1.25 wt %, between 0.5 wt % and 1.25 wt %, between 0.6 wt % and 1.25 wt %, between 0.7 wt % and 1.25 wt %, between 0.8 wt % and 1.25 wt %, between 0.9 wt % and 1 wt %, e.g. 0.05, 0.1, 0.2, 0.25, 0.3, 0.4, 0.5, 0.6, 0.7, 0.8, 0.9, 1, 1.1, 1.2, or 1.25 wt % of a structural protein, and

between 8.75 wt % and 49.95 wt % water, in particular between 9 wt % and 49.9 wt %, between 9 wt % and 49.8 wt %, between 9 wt % and 49.7 wt %, between 9 wt % and 49.6 wt %, between 9 wt % and 49.5 wt %, between 9 wt % and 49.4 wt %, between 9 wt % and 49.3wt %, between 9 wt % and 49.2 wt %, between 9 wt % and 49.1 wt %, between 9 wt % and 49 wt %, between 10 wt % and 48 wt %, between 11 wt % and 47 wt %, between 12 wt % and 46 wt %, between 13 wt % and 45 wt %, between 14 wt % and 44 wt %, between 15 wt % and 43 wt %, between 16 wt % and 42 wt %, between 17 wt % and 41 wt %, between 18 wt % and 40 wt %, between 19 wt % and 39 wt %, between 20 wt % and 38 wt %, between 21 wt % and 37 wt %, between 22 wt % and 36 wt %, between 23 wt % and 35 wt %, between 24 wt % and 34 wt %, between 25 wt % and 33 wt %, between 26 wt % and 32 wt %, between 27 wt % and 31 wt %, or between 28 wt % and 30 wt %, e.g. 8.75, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 48.75, 48.8, 48.9, 49, 49.1, 49.2, 49.3, 49.4, 49.5, 49.6, 49.7, 49.75, 49.8, 49.9, or 49.95 wt % water, wherein the structural protein is the silk protein C₁₆ or are variants thereof.

In one another preferred embodiment, the formulation is a non-flowable hydrogel. The non-flowable hydrogel may also be designated as an aqueous dispersion in a solid state. In the non-flowable hydrogel, the structural protein is preferably present in a concentration of between >1.25 wt % and ≤5 wt %, more preferably present in a concentration of between 1.5 wt % and 1.75 wt %, e.g. in a concentration of 1.26, 1.3, 1.4, 1.5, 1.6, 1.7, 1.75, 1.8, 1.9, 2, 2.5, 3, 3.5, 4, 4.5, 4.6, 4.7, 4.8, 4.9, 4.95, 4.99 wt %, wherein the structural protein is the silk protein C₁₆ or are variants thereof.

In one more preferred embodiment, the non-flowable hydrogel comprises

between 60 wt % and 90 wt % alcohol, in particular between 61 wt % and 89 wt %, between 62 wt % and 88 wt %, between 63 wt % and 87 wt %, between 64 wt % and 86 wt %, between 65 wt % and 85 wt %, between 66 wt % and 84 wt %, between 67 wt % and 83 wt %, between 68 wt % and 82 wt % alcohol, between 69 wt % and 81 wt %, between 70 wt % and 80 wt %, between 71 wt % and 79 wt %, between 72 wt % and 78 wt %, between 73 wt % and 77 wt %, or between 74 wt % and 76 wt % alcohol, e.g. 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 wt % alcohol,

between 5 wt % and 38.75 wt % water, in particular between 5 wt % and 38.7 wt %, between 5 wt % and 38.6 wt %, between 5 wt % and 38.5 wt %, between 5 wt % and 38.4 wt %, between 5 wt % and 38.3 wt %, between 5 wt % and 38.2 wt %, between 5 wt % and 38.1 wt %, between 5 wt % and 38 wt %, between 6 wt % and 37 wt %, between 7 wt % and 36 wt %, between 8 wt % and 35 wt %, between 9 wt % and 34 wt %, between 10 wt % and 33 wt %, between 11 wt % and 32 wt %, between 12 wt % and 31 wt %, between 13 wt % and 30 wt %, between 14 wt % and 29 wt %, between 15 wt % and 28 wt %, between 16 wt % and 27 wt %, between 17 wt % and 26 wt %, between 18 wt % and 25 wt %, between 19 wt % and 24 wt %, between 20 wt % and 23 wt %, or between 21 wt % and 22 wt %, e.g. 5, 5.01, 6, 7, 8, 8.1, 8.2, 8.3, 8.4, 8.5, 8.6, 8.7, 8.74, 8.8, 8.9, 9, 10, 11, 12, 13, 14, 15, 16, 17, 18, 19, 20, 21, 22, 23, 24, 25, 26, 27, 28, 29, 30, 31, 32, 33, 34, 35, 35.01, 35.5, 36, 36.5, 37, 37.5, 38, 38.1, 38.2, 38.3, 38.4, 38.5, 38.6, 38.7, 38.74, or 38.75 water, and

a structural protein in a concentration of >1.25 wt % and ≤5 wt %, in particular 1.3 wt % and 4.5 wt %, 1.4 wt % and 4.5 wt %, 1.5 wt % and 4.5 wt %, 2 wt % and 4 wt %, or 2.5 wt % and 3.5 wt %, e.g. 1.26, 1.3, 1.4, 1.5, 1.6, 1.7, 1.8, 1.9, 2, 2.5, 3, 3.5, 4, 4.5, 4.6, 4.7, 4.8, 4.9, 4.95, 4.99 wt %, wherein the structural protein is the silk protein C₁₆ or are variants thereof.

It is particularly preferred that a hydrogel comprising the silk protein C₈ in a concentration ≤1.625 wt % is a flowable hydrogel and a hydrogel comprising the silk protein C₈ in a concentration >1.625 wt %, e.g. 1.75 wt %, is a non-flowable hydrogel.

It is particularly preferred that a hydrogel comprising the silk protein C₁₆ in a concentration ≤1.25 wt % is a flowable hydrogel and a hydrogel comprising the silk protein C₁₆ in a concentration >1.25 wt %, e.g. 1.5 wt % and 2.0 wt %, is a non-flowable hydrogel.

It is particularly more preferred that a flowable hydrogel comprises the silk protein C₁₆ in a concentration of between 0.05 wt % and ≤1.25 wt %.

It is particularly more preferred that a non-flowable hydrogel comprises the silk protein C₁₆ in a concentration of between >1.25 wt % and ≤5 wt %.

It is further particularly preferred that a hydrogel comprising the silk protein C₃₂ in a concentration ≤0.75 wt % is a flowable hydrogel and a hydrogel comprising the silk protein C₃₂ in a concentration >0.75 wt %, e.g. of 1.0 wt % and 1.25 wt %, is a non-flowable hydrogel.

It is particularly more preferred that a flowable hydrogel comprises the silk protein C₃₂ in a concentration of between 0.05 wt % and ≤0.75 wt %.

It is particularly more preferred that a non-flowable hydrogel comprises the silk protein C₃₂ in a concentration of between >0.75 wt % and ≤5 wt %.

It is also particularly preferred that a hydrogel comprising the silk protein C₄₈ in a concentration ≤0.5 wt % is a flowable hydrogel and a hydrogel comprising a protein concentration >0.5 wt %, e.g. 0.75 wt %, 1.0 wt %, or 1.165 wt % is a non-flowable hydrogel.

It is particularly more preferred that a flowable hydrogel comprises the silk protein C₄₈ in a concentration of between 0.05 wt % and ≤0.5 wt %.

It is particularly more preferred that a non-flowable hydrogel comprises the silk protein C₄₈ in a concentration of between >0.5 wt % and ≤5 wt %.

The silk proteins C₈, C₁₆, C₃₂, or C₄₈ mentioned above also encompass variants thereof.

The structural protein is preferably a self-assembling protein. Said self-assembling protein has the potential to self-assemble into fibrillary structures (i.e. fibrillary complexes of structural proteins).

It is further preferred that the structural protein is selected from the group consisting of a silk protein, keratin, collagen, and elastin. In particular, the (self-assembling) structural protein is a recombinant protein, e.g. a recombinant silk protein, keratin, collagen, or elastin.

It is more preferred that the (self-assembling) structural protein is a silk protein, e.g. a recombinant silk protein. The (recombinant) silk protein may be a spider silk protein, e.g. a major ampullate silk protein such as a dragline silk protein, a minor ampullate silk protein, or a flagelliform silk protein of an orb-web spider (Preferably, the silk protein is a spider silk protein, more preferably a recombinant spider silk protein.

It is further (alternatively or additionally) more preferred that the silk protein is a protein with an amino acid sequence which comprises or consists of at least 50%, 60%, 65%, 70%, 75%, 80%, 85%, or 90% multiple copies of repetitive units. It is even more preferred that the silk protein is a protein with an amino acid sequence which comprises or consists of at least 95% multiple copies of repetitive units. Said repetitive units may be identical or different.

It is particularly preferred that the silk protein comprises at least two identical repetitive units. For example, the silk protein may comprise between 2 to 100 repetitive units, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 repetitive units.

It is also (alternatively or additionally) more preferred that the silk protein consists of between 40 to 3000 amino acids. It is even more preferred that the silk protein consists of between 40 to 1500 amino acids or between 200 to 1200 amino acids. It is most preferred that the silk protein consists of between 250 to 600 amino acids.

It is further particularly preferred that the silk protein comprises at least two identical repetitive units. In one embodiment, the repetitive units are independently selected from the group consisting of module C (SEQ ID NO: 1) or a variant thereof and module C^{Cys} (said module may also be designated as module C^{C}) (SEQ ID NO: 2). Module C^{Cys} (SEQ ID NO: 2) is a variant of module C (SEQ ID NO: 1). In this module, the amino acid S (Ser) at position 25 has been replaced by the amino acid C (Cys).

The module C variant differs from the reference module C from which it is derived by up to 1, 2, 3, 4, 5, 6, 7, 8, 9, 10, 11, 12, 13, 14, or 15 amino acid changes in the amino acid sequence (i.e. substitutions, additions, insertions, deletions, N-terminal truncations and/or C-terminal truncations). Such a module variant can alternatively or additionally be characterised by a certain degree of sequence identity to the reference module from which it is derived. Thus, the module C variant has a sequence identity of at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or even 99.9% to the respective reference module C. Preferably, the sequence identity is over a continuous stretch of at least 5, 10, 15, 18, 20, 24, 27, 28, 30, 34, 35, or more amino acids, preferably over the whole length of the respective reference module C.

The sequence identity may be at least 80% over the whole length, may be at least 85% over the whole length, may be at least 90% over the whole length, may be at least 95% over the whole length, may be at least 98% over the whole length, or may be at least 99% over the whole length of the respective reference module C. Alternatively, the sequence identity may be at least 80% over a continuous stretch of at least 5, 10, 15, 18, 20, 24, 28, or 30 amino acids, may be at least 85% over a continuous stretch of at least 5, 10, 15, 18, 20, 24, 28, or 30 amino acids, may be at least 90% over a continuous stretch of at least 5, 10, 15, 18, 20, 24, 28, or 30 amino acids, may be at least 95% over a continuous stretch of at least 5, 10, 15, 18, 20, 24, 28, or 30 amino acids, may be at least 98% over a continuous stretch of at least 5, 10, 15, 18, 20, 24, 28, or 30 amino acids, or may be at least 99% over a continuous stretch of at least 5, 10, 15, 18, 20, 24, 28, or 30 amino acids of the respective reference module C.

A fragment (or deletion) variant of module C has preferably a deletion of up to 1, 2, 3, 4, 5, 6, 7, 8, 9, 10, 11, 12, 13, 14, or 15 amino acids at its N-terminus and/or at its C-terminus. The deletion can also be internally.

Additionally, the module C variant or fragment is only regarded as a module C variant or fragment within the context of the present invention, if the modifications with respect to the amino acid sequence on which the variant or fragment is based do not negatively affect the ability of the silk polypeptide to form, together with an alcohol, an aqueous formulation, in particular a hydrogel, e.g. a flowable hydrogel or a non-flowable hydrogel, comprising a structural protein and an alcohol. The skilled person can readily assess whether the silk polypeptide comprising a module C variant or fragment is still capable of forming, together with an alcohol, an aqueous formulation, in particular a hydrogel, e.g. a flowable hydrogel or a non-flowable hydrogel, comprising a structural protein and an alcohol. In this respect, it is referred to the examples comprised in the experimental part of the present patent application. C^{Cys} variants may also be encompassed by the present invention. Regarding the C^{Cys} variants, the same explanations/definitions apply which have been made with respect to the module C variant (see above).

Preferably, the silk polypeptide is selected from the group consisting of (C)ₘ, (C^{Cys})ₘ, (C)ₘC^{Cys}, C^{Cys}(C)ₘ, (C)ₘC^{Cys}(C)ₘ, wherein m is an integer of 8 to 96, i.e. 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61,62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95 or 96.

More preferably, the silk polypeptide is selected from the group consisting of C₈, C₁₆, C₃₂, C₄₈, C₈C^{Cys}, C₁₆C^{Cys}, C₃₂C^{Cys}, C₄₈C^{Cys}, C^{Cys}C₈, C^{Cys}C₁₆, C^{Cys}C₃₂, and C^{Cys}C₄₈.

It is also preferred that said formulation, preferably the hydrogel, more preferably the flowable hydrogel or the non-flowable hydrogel, further comprises a compound. The compound may be poorly water soluble, water insoluble, lipophilic, or oily. The compound may further be selected from the group consisting of a pharmaceutical compound, a detergent compound, a cosmetic compound, a chemical compound and a coloring compound. The detergent compound may be a cleaning agent or a laundry detergent. The cosmetic compound may be a fragrance oil or fragrance. The coloring compound may be a dye.

In a second aspect, the present invention relates to a method for producing an aqueous formulation comprising a structural protein and an alcohol comprising the steps of:
(i) providing an aqueous solution comprising a structural protein and an aqueous solution comprising an alcohol, and
(ii) mixing the aqueous solutions (provided in step (i)), thereby obtaining an aqueous formulation comprising a structural protein and an alcohol.

In one embodiment, the method further comprises subsequent to step (i) a step of adding the aqueous solution comprising an alcohol to the aqueous solution comprising a structural protein.

Thus, in one embodiment, the method for producing an aqueous formulation comprising a structural protein and an alcohol comprises the steps of:
(i) providing an aqueous solution comprising a structural protein and an aqueous solution comprising an alcohol,
(ii) adding the aqueous solution comprising an alcohol to the aqueous solution comprising a structural protein, and
(iii) mixing the aqueous solutions, thereby obtaining an aqueous formulation comprising a structural protein and an alcohol.

The addition of the aqueous solution comprising an alcohol to the aqueous solution comprising a structural protein is preferably performed by pouring, titrating, or dripping the aqueous solution comprising an alcohol to/into the aqueous solution comprising a structural protein. The present inventors have surprisingly found that the addition of an aqueous solution comprising an alcohol to an aqueous solution comprising a structural protein, in particular by pouring, titrating, or dripping, results in an aqueous formulation having a clear appearance and/or comprising no visible aggregates and/or precipitates. In contrast thereto, the aqueous formulation described in the prior art is turbid and comprises visible aggregates and/or precipitates. In the prior art, alcohol is often used as an aggregation trigger. Thus, it was very surprising for the present inventors that the above preparation process results in an aqueous formulation having a clear appearance and/or comprising no visible aggregates and/precipitates.

It is preferred that the aqueous solution comprising an alcohol is added to the aqueous solution comprising a structural protein, in particular by pouring, titrating, or dripping, in one motion/at once, more preferred in one motion/at once as fast as possible. In another preferred embodiment, the aqueous solution comprising an alcohol is added to the aqueous solution comprising a structural protein, in particular by pouring, titrating, or dripping, in one motion/at once within no more than 60 seconds, preferably within no more than 20 seconds, more preferably within no more than 10 seconds, e.g. within no more than 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 seconds. The present inventors have surprisingly found that the addition of the aqueous solution comprising an alcohol to the aqueous solution comprising a structural protein, in particular by pouring, titrating, or dripping, in this way/in this order prevents the formation of visible aggregates and/or precipitates in the resulting aqueous formulation.

The mixing step is preferably performed immediately after the addition of the aqueous solution comprising an alcohol to the aqueous solution comprising a structural protein. For example, the mixing step is started no more than 10 seconds, e.g. no more than 1, 2, 3, 4, 5, 6, 7, 8, 9, or 10 seconds, after the addition of the aqueous solution comprising an alcohol to the aqueous solution comprising a structural protein. It is preferred that the aqueous solutions are mixed until a homogenous aqueous formulation comprising a structural protein and an alcohol is reached. The mixing step is preferably performed as fast as possible. In another preferred embodiment, the aqueous solutions are mixed for no more than 60 seconds, preferably for no more than 20 seconds, more preferably for no more than 10 seconds, e.g. for no more than 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 seconds. The mixing step results in an aqueous formulation in which the structural protein and the alcohol are preferably homogenously distributed.

The mixing is preferably performed by avoiding the application of sheer forces, preferably by (gently) agitating, (gently) stirring, or (gently) swiveling. For example, the mixing may be performed in a static mixer. The static mixer allows the continuous mixing of the aqueous solution comprising a structural protein and the aqueous solution comprising an alcohol. For large-scale production, the mixing may be performed by (gently) stirring. The mixing results in an aqueous formulation in which the structural protein and the alcohol are preferably homogenously distributed.

This embodiment is further described as option 1 in the examples/figures of the present patent application.

In one alternative embodiment, the method further comprises subsequent to step (i) a step of (simultaneously) bringing together/combining the aqueous solution comprising a structural protein and the aqueous solution comprising an alcohol.

Thus, in one alternative embodiment, the method for producing an aqueous formulation comprising a structural protein and an alcohol comprises the steps of:
(i) providing an aqueous solution comprising a structural protein and an aqueous solution comprising an alcohol, and
(ii) (simultaneously) bringing together/combining the aqueous solution comprising an alcohol and the aqueous solution comprising a structural protein, and
(iii) mixing the aqueous solutions, thereby obtaining an aqueous formulation comprising a structural protein and an alcohol.

The simultaneous merger/combination of the aqueous solution comprising an alcohol and the aqueous solution comprising a structural protein is preferably performed by simultaneously pouring both solutions into a container. In particular, the simultaneous merger/combination of the aqueous solution comprising an alcohol and the aqueous solution comprising a structural protein is performed by pouring both solutions into a container such that both solutions come in contact with each other, e.g. at the container bottom and/or before they hit the container bottom.

The present inventors have surprisingly found that the simultaneous merger/combination of the aqueous solution comprising an alcohol and the aqueous solution comprising a structural protein, in particular by pouring both solutions into a container, prevents the formation of visible aggregates and/or precipitates in the resulting aqueous formulation.

The mixing step is preferably performed once the solutions are in contact with each other. It is preferred that the aqueous solutions are mixed until a homogenous aqueous formulation comprising a structural protein and an alcohol is reached. The mixing step is preferably performed as fast as possible. In another preferred embodiment, the aqueous solutions are mixed for no more than 60 seconds, preferably for no more than 20 seconds, more preferably for no more than 10 seconds, e.g. for no more than 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 seconds. The mixing step results in an aqueous formulation in which the structural protein and the alcohol are preferably homogenously distributed.

The mixing is preferably performed by (rapid) stirring or (rapid) agitating. For example, the mixing may be performed in a static mixer or using an agitator. The static mixer or agitator allows the continuous mixing of the aqueous solution comprising a structural protein and the aqueous solution comprising an alcohol. For large-scale production the mixing may be performed by stirring.

This embodiment is further described as option 2 in the examples/figures of the present patent application.

In one alternative embodiment, the method further comprises subsequent to step (i) a step of undercoating/underlayering the aqueous solution comprising an alcohol with the aqueous solution comprising a structural protein.

Thus, in one alternative embodiment, the method for producing an aqueous formulation comprising a structural protein and an alcohol comprises the steps of:
(i) providing an aqueous solution comprising a structural protein and an aqueous solution comprising an alcohol, and
(ii) undercoating/underlayering the aqueous solution comprising an alcohol with/by the aqueous solution comprising a structural protein, and
(iii) mixing the aqueous solutions, thereby obtaining an aqueous formulation comprising a structural protein and an alcohol.

The undercoating/underlayering of the aqueous solution comprising an alcohol with the aqueous solution comprising a structural protein is preferably performed by introducing an aqueous solution comprising a structural protein below the surface of the aqueous solution comprising an alcohol. For this purpose, the aqueous solution comprising an alcohol is preferably comprised in a container and the container is preferably designed as having an inlet. The inlet is arranged below the filling level of the aqueous solution comprising an alcohol so that when the aqueous solution comprising a structural protein is introduced into the container trough the inlet, it enters the container at a position below the surface of the aqueous solution comprising an alcohol.

In particular, the undercoating/underlayering of the aqueous solution comprising an alcohol with the aqueous solution comprising a structural protein results in a two-phase liquid system comprising an upper alcohol containing aqueous solution phase and an under/a base structural protein containing aqueous solution phase. Due to the differences in density (the aqueous solution comprising a structural protein has a higher density than the aqueous solution comprising an alcohol), the two-phase liquid system is produced.

The present inventors have surprisingly found that the undercoating/underlayering of the aqueous solution comprising an alcohol with the aqueous solution comprising a structural protein prevents the formation of visible aggregates and/or precipitates in the resulting aqueous formulation.

When both phases are then mixed with each other, an aqueous formulation comprising a structural protein and an alcohol is formed. The mixing step is preferably performed after the formation of the two-phase liquid system. It is preferred that the aqueous solutions/phases are mixed until a homogenous aqueous formulation comprising a structural protein and an alcohol is reached. The mixing step is preferably performed as fast as possible. In another preferred embodiment, the aqueous solutions are mixed for no more than 60 seconds, preferably for no more than 20 seconds, more preferably for no more than 10 seconds, e.g. for no more than 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 seconds. The mixing step results in an aqueous formulation in which the structural protein and the alcohol are preferably homogenously distributed.

The mixing is preferably performed by (rapid) stirring or (rapid) agitating. For example, the mixing may be performed in a mixer or using an agitator. The mixer or agitator allows the continuous mixing of the aqueous solution comprising a structural protein and the aqueous solution comprising an alcohol. For large-scale production the mixing may be performed by stirring.

This embodiment is further described as option 3 in the examples/figures of the present patent application.

It is further preferred that the concentration of the structural protein in the aqueous solution provided in step (i) is of between 0.05 wt % and 5 wt %, preferably of between 0.5 wt % and 3 wt %, and more preferably of between 0.75 wt % and 2 wt %. For example, the concentration of the structural protein in the aqueous solution provided in (i) is 0.05, 0.1, 0.2, 0.3, 0.4, 0.5, 0.6, 0.7, 0.8, 0.9, 1, 1.1, 1.175, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 wt %. In particular, the structural protein is present in the aqueous solution in a concentration of between 0.05 wt % and 5 wt %, in particular of between 0.1 wt % and 5 wt %, between 0.2 wt % and 5 wt %, between 0.3 wt % and 5 wt %, between 0.4 wt % and 5 wt %, between 0.5 wt % and 5 wt %, between 0.6 wt % and 5 wt %, between 0.7 wt % and 5 wt %, between 0.8 wt % and 5 wt %, between 0.9 wt % and 5 wt %, between 1 wt % and 5 wt %, between 1.5 wt % and 4.5 wt %, between 2 wt % and 4 wt %, or between 2.5 wt % and 3.5 wt %.

The structural protein may be the silk protein C₈, C₁₆, C₃₂, C₄₈, or variants thereof.

It is further preferred that the concentration of the alcohol in the aqueous solution provided in step (i) is of between 50 wt % and 90 wt %, preferably of between 65 wt % and 85 wt %, and more preferably of between 70 wt % and 80 wt %. For example, the concentration of the alcohol in the aqueous solution added in step (ii) is 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 wt %.

In particular, the aqueous solution comprising a structural protein is homogenous. In this respect, homogenous means that the structural protein is dispersed in the aqueous solution.

In one embodiment, the aqueous formulation comprising a structural protein and an alcohol which is produced in step (ii/iii) is a hydrogel comprising a structural protein and an alcohol.

In one preferred embodiment, the aqueous formulation comprising a structural protein and an alcohol which is produced in step (ii/iii) is a flowable hydrogel comprising a structural protein and an alcohol. In case that a flowable hydrogel comprising a structural protein and an alcohol is produced in step (ii/iii), the concentration of the structural protein in the aqueous solution provided in (i) is preferably of between 0.05 wt % and 1.25 wt %, more preferably of between 0.75 wt % and 1.25 wt %, wherein the structural protein is the silk protein C₁₆ or are variants thereof. For example, the concentration of the structural protein in the aqueous solution provided in (i) is 0.05, 0.1, 0.2, 0.25, 0.3, 0.4, 0.5, 0.6, 0.7, 0.75, 0.8, 0.9, 1, 1.1, 1.2, or 1.25 wt %, wherein the structural protein is the silk protein C₁₆ or are variants thereof.

In one another preferred embodiment, the aqueous formulation comprising a structural protein and an alcohol which is produced in step (ii/iii) is a non-flowable hydrogel comprising a structural protein and an alcohol. In case that a non-flowable hydrogel comprising a structural protein and an alcohol is produced in step (ii/iii), the concentration of the structural protein in the aqueous solution provided in (i) is preferably >1.25 wt % and ≤5 wt %, more preferably 1.5 wt % and 1.75 wt %, wherein the structural protein is the silk protein C₁₆ or are variants thereof. For example, the concentration of the structural protein in the aqueous solution provided in (i) is 1.26, 1.3, 1.4, 1.5, 1.6, 1.7, 1.75, 1.8, 1.9, 2, 2.5, 3, 3.5, 4, 4.5, 4.6, 4.7, 4.8, 4.9, 4.95, 4.99 wt %, wherein the structural protein is the silk protein C₁₆ or are variants thereof.

It is particularly preferred that a hydrogel which is produced in step (ii/iii) and which comprises the silk protein C₈ in a concentration ≤1.625 wt % is a flowable hydrogel and a hydrogel which is produced in step (ii/iii) and which comprises the silk protein C₈ in a concentration >1.625 wt %, e.g. 1.75 wt %, is a non-flowable hydrogel.

It is particularly preferred that a hydrogel which is produced in step (ii/iii) and which comprises the silk protein C₁₆ in a concentration ≤1.25 wt % is a flowable hydrogel and a hydrogel which is produced in step (ii/iii) and which comprises the silk protein C₁₆ in a concentration >1.25 wt %, e.g. 1.5 wt % and 2.0 wt %, is a non-flowable hydrogel.

It is particularly more preferred that a flowable hydrogel which is produced in step (ii/iii) comprises the silk protein C₁₆ in a concentration of between 0.05 wt % and ≤1.25 wt %.

It is particularly more preferred that a non-flowable hydrogel which is produced in step (ii/iii) comprises the silk protein C₁₆ in a concentration of between >1.25 wt % and ≤5 wt %.

It is further particularly preferred that a hydrogel which is produced in step (ii/iii) and which comprises the silk protein C₃₂ in a concentration ≤0.75 wt % is a flowable hydrogel and a hydrogel which is produced in step (ii/iii) and which comprises the silk protein C₃₂ in a concentration >0.75 wt %, e.g. of 1.0 wt % and 1.25 wt %, is a non-flowable hydrogel.

It is particularly more preferred that a flowable hydrogel which is produced in step (ii/iii) comprises the silk protein C₃₂ in a concentration of between 0.05 wt % and ≤0.75 wt %.

It is particularly more preferred that a non-flowable hydrogel which is produced in step (ii/iii) comprises the silk protein C₃₂ in a concentration of between >0.75 wt % and ≤5 wt %.

It is also particularly preferred that a hydrogel which is produced in step (ii/iii) and which comprises the silk protein C₄₈ in a concentration ≤0.5 wt % is a flowable hydrogel and a hydrogel which is produced in step (ii/iii) and which comprises a protein concentration >0.5 wt %, e.g. 0.75 wt %, 1.0 wt %, or 1.165 wt % is a non-flowable hydrogel.

It is particularly more preferred that a flowable hydrogel which is produced in step (ii/iii) comprises the silk protein C₄₈ in a concentration of between 0.05 wt % and ≤0.5 wt %.

It is particularly more preferred that a non-flowable hydrogel which is produced in step (ii/iii) comprises the silk protein C₄₈ in a concentration of between >0.5 wt % and ≤5 wt %.

The silk proteins C₈, C₁₆, C₃₂, or C₄₈ mentioned above also encompass variants thereof.

The alcohol may be selected from the group consisting of ethanol, methanol, and isopropanol. The ethanol may be ethanol having a purity of ≥99.5% (p.a.).

Preferably, the structural protein has a molecular weight of between 20 kDa and 140 kDa, more preferably of between 20 kDa and 95 kDa or between 30 kDa and 75 kDa, and even more preferably of between 40 kDa and 55 kDa. For example, the structural protein has a molecular weight of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, or 140 kDa.

It is also preferred that the method further comprises the step of adding a compound to

the aqueous solution comprising a structural protein provided in step (i),

the aqueous solution comprising an alcohol provided in step (i), and/or

the mixture in step (ii/iii).

The compound may be poorly water soluble, water insoluble, lipophilic, or oily. The compound may further be selected from the group consisting of a pharmaceutical compound, a detergent compound, a cosmetic compound, a chemical compound and a coloring compound. The detergent compound may be a cleaning agent or a laundry detergent. The cosmetic compound may be a fragrance oil or fragrance. The coloring compound may be a dye.

The structural protein is preferably a self-assembling protein. Said self-assembling protein has the potential to self-assemble into fibrillary structures.

It is further preferred that the structural protein is selected from the group consisting of a silk protein, keratin, collagen, and elastin. In particular, the (self-assembling) structural protein is a recombinant protein, e.g. a recombinant silk protein, keratin, collagen, or elastin.

It is more preferred that the (self-assembling) structural protein is a silk protein, e.g. a recombinant silk protein. The (recombinant) silk protein may be a spider silk protein, e.g. a major ampullate silk protein such as a dragline silk protein, a minor ampullate silk protein, or a flagelliform silk protein of an orb-web spider Preferably, the silk protein is a spider silk protein, more preferably a recombinant spider silk protein.

It is further (alternatively or additionally) more preferred that the silk protein is a protein with an amino acid sequence which comprises or consists of at least 50%, 60%, 65%, 70%, 75%, 80%, 85%, or 90% multiple copies of repetitive units. It is even more preferred that the silk protein is a protein with an amino acid sequence which comprises or consists of at least 95% multiple copies of repetitive units. Said repetitive units may be identical or different.

It is particularly preferred that the silk protein comprises at least two identical repetitive units. For example, the silk protein may comprise between 2 to 100 repetitive units, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 repetitive units.

It is also (alternatively or additionally) more preferred that the silk protein consists of between 40 to 3000 amino acids. It is even more preferred that the silk protein consists of between 40 to 1500 amino acids or between 200 to 1200 amino acids. It is most preferred that the silk protein consists of between 250 to 600 amino acids.

It is further particularly preferred that the silk protein comprises at least two identical repetitive units. In one embodiment, the repetitive units are independently selected from the group consisting of module C (SEQ ID NO: 1) or a variant thereof and module C^{Cys} (said module may also be designated as module C^{C}) (SEQ ID NO: 2). Module C^{Cys} (SEQ ID NO: 2) is a variant of module C (SEQ ID NO: 1). In this module, the amino acid S (Ser) at position 25 has been replaced by the amino acid C (Cys).

As to the module C variant or module C^{Cys} variant, it is referred to the first aspect of the present invention.

Preferably, the silk polypeptide is selected from the group consisting of (C)ₘ, (C^{Cys})ₘ, (C)ₘC^{Cys}, C^{Cys}(C)ₘ, (C)ₘC^{Cys}(C)ₘ, wherein m is an integer of 8 to 96, i.e. 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61,62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95 or 96.

More preferably, the silk polypeptide is selected from the group consisting of C₈, C₁₆, C₃₂, C₄₈, C₈C^{Cys}, C₁₆C^{Cys}, C₃₂C^{Cys}, C₄₈C^{Cys}, C^{Cys}C₈, C^{Cys}C₁₆, C^{Cys}C₃₂, and C^{Cys}C₄₈.

In a third aspect, the present invention relates to an aqueous formulation comprising a structural protein and an alcohol obtainable by the method according to the second aspect.

In one embodiment, the aqueous formulation comprising a structural protein and an alcohol is a hydrogel comprising a structural protein and an alcohol.

In one preferred embodiment, the aqueous formulation comprising a structural protein and an alcohol is a flowable hydrogel comprising a structural protein and an alcohol.

In one another preferred embodiment, the aqueous formulation comprising a structural protein and an alcohol is a non-flowable hydrogel comprising a structural protein and an alcohol.

In a fourth aspect, the present invention relates to a method for producing an article comprising the steps of:
(i) providing an aqueous formulation comprising a structural protein and an alcohol according to the first or third aspect, and
(ii) forming an article out of/from the formulation provided in (i).

The article may be selected from the group consisting of a film, a coating, a particle, a capsule, a fiber, and a porous structure such as a scaffold or a foam.

In one embodiment, the aqueous formulation comprising a structural protein and an alcohol is a hydrogel comprising a structural protein and an alcohol.

In one preferred embodiment, the aqueous formulation comprising a structural protein and an alcohol is a flowable hydrogel comprising a structural protein and an alcohol. In this case, the method for producing an article comprises the steps of:
(i) providing a flowable hydrogel comprising a structural protein and an alcohol according to the first or third aspect, and
(ii) forming an article out of/from the flowable hydrogel provided in (i).

The article may be selected from the group consisting of a fiber, a film, or a coating.

In one embodiment, the article is a fiber.

When the article which is produced is a fiber, step (ii) comprises drawing a fiber from the flowable hydrogel comprising a structural protein and an alcohol according to the first or third aspect, or extruding and drawing a fiber from the flowable hydrogel comprising a structural protein and an alcohol according to the first or third aspect. Thus, in one embodiment, the method is for producing a fiber and comprises the steps of:
(i) providing a flowable hydrogel comprising a structural protein and an alcohol according to the first or third aspect, and
(ii) forming an article out of/from the flowable hydrogel provided in (i), wherein said formation comprises drawing a fiber from the flowable hydrogel comprising a structural protein and an alcohol, or extruding and drawing a fiber from the flowable hydrogel comprising a structural protein and an alcohol.

Spinning methods such as wet spinning or electrospinning methods are known to the skilled person. For example, the flowable hydrogel is extruded through a spinneret to form a fiber.

The fiber may be used to make a fabric, e.g. a woven or non-woven fabric. The skilled person is aware of techniques allowing to generate a fabric, e.g. weaving processes. Thus, in an alternative embodiment, the article may be a fabric made of fibers.

In one another embodiment, the article is a film.

When the article which is produced is a film, step (ii) comprises casting or spraying a flowable hydrogel comprising a structural protein and an alcohol according to the first or third aspect onto a substrate.

Thus, in one another embodiment, the method is for producing a film and comprises the steps of:
(i) providing a flowable hydrogel comprising a structural protein and an alcohol according to the first or third aspect, and
(ii) forming an article out of/from the flowable hydrogel provided in (i), wherein said formation comprises casting or spraying the flowable hydrogel comprising a structural protein and an alcohol onto a substrate.

In one further (alternatively or additionally) preferred embodiment, the method further comprises the step of:
(iii) drying the film.

In one further (alternatively or additionally) preferred embodiment, the method further comprises the step of:
(iv) separating/removing the film from the substrate.

When the article is a coating, the same methods steps (i), (ii) and (iii) as for the production of a film apply.

In one another preferred embodiment, the aqueous formulation comprising a structural protein and an alcohol is a non-flowable hydrogel comprising a structural protein and an alcohol. In this case, the method for producing an article comprises the steps of:
(i) providing a non-flowable hydrogel comprising a structural protein and an alcohol according to the first or third aspect, and
(ii) forming an article out of/from the non-flowable hydrogel provided in (i).

The article can be used to fill cavities or in tissue engineering. In particular, the non-flowable hydrogel can be converted into a flowable hydrogel using energy input in form of application of sheer forces. Therefore, the non-flowable hydrogel can be, for example, extruded through a nozzle In addition, the non-flowable hydrogel can be nebulized with the help of an ultrasonic device to liquify the hydrogel provided in (i). Out of/from the resulting flowable hydrogel, an article can be formed. The article may be selected from the group consisting of a fiber, a film, or a coating.

Preferably, the method further comprises the step of adding a compound to the aqueous formulation provided in step (i) or to the article formed in step (ii). The aqueous formulation provided in step (i) may be a hydrogel.

In one preferred embodiment, the aqueous formulation provided in step (i) is a flowable hydrogel comprising a structural protein and an alcohol.

In one another preferred embodiment, the aqueous formulation provided in step (i) is a non-flowable hydrogel comprising a structural protein and an alcohol.

When the aqueous formulation is a flowable hydrogel comprising a structural protein and an alcohol, the compound may be added to the flowable hydrogel by mixing the compound with the flowable hydrogel prior to forming the article. The compound may also be loaded into the article or coated onto the article after it is formed from the flowable hydrogel comprising a structural protein and an alcohol.

When the aqueous formulation is a non-flowable hydrogel comprising a structural protein and an alcohol, the compound may be added to the non-flowable hydrogel by loading the compound into the non-flowable hydrogel prior to forming the article. The compound may also loaded into the article or coated onto the article after it is formed from the non-flowable hydrogel comprising a structural protein and an alcohol.

The compound may be poorly water soluble, water insoluble, lipophilic, or oily. The compound may further be selected from the group consisting of a pharmaceutical compound, a detergent compound, a cosmetic compound, a chemical compound, and a coloring compound. The detergent compound may be a cleaning agent or a laundry detergent. The cosmetic compound may be a fragrance oil or fragrance. The coloring compound may be a dye.

In a fifth aspect, the present invention relates to an article obtainable by the method according to the fourth aspect.

The article may be selected from the group consisting of a film, a coating, a particle, a capsule, a fiber, and a porous structure such as a scaffold or a foam.

In a sixth aspect, the present invention relates to a pharmaceutical composition comprising

the aqueous formulation comprising a structural protein and an alcohol according to the first or third aspect, or

the article according to the fifth aspect.

In particular, the pharmaceutical composition (in particular the aqueous formulation or the article) comprises a pharmaceutical compound. The pharmaceutical composition may also comprise pharmaceutical acceptable carriers, diluents, and/or excipients. The pharmaceutical composition is administered to a patient. It is useful for treating, preventing, or reducing the severity of a disease or disorder in the patient. It may be administered locally or systemically to the patient. The local administration may be by parenteral administration, e.g. intravenous administration, subcutaneous administration, intradermal administration, or intramuscularly administration. The systemic administration may be by intraarterial administration.

In one embodiment, the aqueous formulation comprising a structural protein and an alcohol is a hydrogel comprising a structural protein and an alcohol. In this case, the pharmaceutical composition comprises

the hydrogel comprising a structural protein and an alcohol according to the first or third aspect, or

the article according to the fifth aspect.

In one preferred embodiment, the aqueous formulation comprising a structural protein and an alcohol is a flowable hydrogel comprising a structural protein and an alcohol. In this case, the pharmaceutical composition comprises

the flowable hydrogel comprising a structural protein and an alcohol according to the first or third aspect, or

the article according to the fifth aspect.

In one another preferred embodiment, the aqueous formulation comprising a structural protein and an alcohol is a non-flowable hydrogel comprising a structural protein and an alcohol. In this case, the pharmaceutical composition comprises

the non-flowable hydrogel comprising a structural protein and an alcohol according to the first or third aspect, or

the article according to the fifth aspect.

In a seventh aspect, the present invention relates to a cosmetic composition comprising

the aqueous formulation comprising a structural protein and an alcohol according to the first or third aspect, or

the article according to the fifth aspect.

In particular, the cosmetic composition (in particular the aqueous formulation or the article) comprises a cosmetic compound. The cosmetic compound may be a fragrance oil or fragrance.

In one embodiment, the aqueous formulation comprising a structural protein and an alcohol is a hydrogel comprising a structural protein and an alcohol. In this case, the cosmetic composition comprises

the hydrogel comprising a structural protein and an alcohol according to the first or third aspect, or

the article according to the fifth aspect.

In one preferred embodiment, the aqueous formulation comprising a structural protein and an alcohol is a flowable hydrogel comprising a structural protein and an alcohol. In this case, the cosmetic composition comprises

the flowable hydrogel comprising a structural protein and an alcohol according to the first or third aspect, or

the article according to the fifth aspect.

In one another preferred embodiment, the aqueous formulation comprising a structural protein and an alcohol is a non-flowable hydrogel comprising a structural protein and an alcohol. In this case, the cosmetic composition comprises

The non-flowable hydrogel comprising a structural protein and an alcohol according to the first or third aspect, or

the article according to the fifth aspect.

In an eight aspect, the present invention relates to an aqueous formulation comprising a structural protein and an alcohol according to the first or third aspect, or

an article according the fifth aspect.

for use as a pharmaceutical.

In particular, the aqueous formulation or article comprises a pharmaceutical compound.

In one embodiment, the aqueous formulation comprising a structural protein and an alcohol is a hydrogel comprising a structural protein and an alcohol. In this case,

the hydrogel comprising a structural protein and an alcohol according to the first or third aspect, or

the article according the fifth aspect

is for use as a pharmaceutical.

In one preferred embodiment, the aqueous formulation comprising a structural protein and an alcohol is a flowable hydrogel comprising a structural protein and an alcohol. In this case,

the flowable hydrogel comprising a structural protein and an alcohol according to the first or third aspect, or

the article according the fifth aspect

is for use as a pharmaceutical.

In one another preferred embodiment, the aqueous formulation comprising a structural protein and an alcohol is a non-flowable hydrogel comprising a structural protein and an alcohol. In this case,

the non-flowable hydrogel comprising a structural protein and an alcohol according to the first or third aspect, or

the article according the fifth aspect

is for use as a pharmaceutical.

In a ninth aspect, the present invention relates to the use of

the aqueous formulation comprising a structural protein and an alcohol according to the first or third aspect, or

the article according to the fifth aspect

for the protection of a compound.

In particular, the aqueous formulation or article comprises a compound. The compound may be selected from the group consisting of a pharmaceutical compound, a detergent compound, a cosmetic compound, a chemical compound, and a coloring compound.

The present inventors have noted that the aqueous formulation is suitable for the protection of a compound against proteolytic degradation, microbial degradation or against oxidation of a compound.

In one embodiment, the aqueous formulation comprising a structural protein and an alcohol is a hydrogel comprising a structural protein and an alcohol. In this case,

the hydrogel comprising a structural protein and an alcohol according to the first or third aspect, or

the article according the fifth aspect

is used for the protection of a compound.

In one preferred embodiment, the aqueous formulation comprising a structural protein and an alcohol is a flowable hydrogel comprising a structural protein and an alcohol. In this case,

the flowable hydrogel comprising a structural protein and an alcohol according to the first or third aspect, or

the article according the fifth aspect

is used for the protection of a compound.

In one another preferred embodiment, the aqueous formulation comprising a structural protein and an alcohol is a non-flowable hydrogel comprising a structural protein and an alcohol. In this case,

the non-flowable hydrogel comprising a structural protein and an alcohol according to the first or third aspect, or

the article according the fifth aspect

is used for the protection of a compound.

In a tenth aspect, the present invention relates to the use of

the aqueous formulation comprising a structural protein and an alcohol according to the first or third aspect, or

the article according to the fifth aspect

for sustained or controlled release of a compound.

In particular, the aqueous formulation or article comprises a compound. The compound may be selected from the group consisting of a pharmaceutical compound, a detergent compound, a cosmetic compound, a chemical compound, and a coloring compound.

The present inventors have noted that the aqueous formulation is suitable for sustained or controlled release of a compound.

Sustained or controlled release refers to the gradual release of the compound from the aqueous formulation over a period of time. While there may be an initial burst phase, it is preferred that the release display relatively linear kinetics, thereby providing a constant supply of the compound over the release period. The release period may vary from several hours to several months, depending upon the properties of the compound and its intended use. For example, it can be desirable that the cumulative release of a compound from the aqueous formulation over a certain period be relatively high to avoid the need for excessive loading of the aqueous formulation and consequent waste of unreleased compound.

It is preferred that the release profile of the aqueous formulation has a sustained release within the first 24 hour. It is also preferred that up to 100% of the compound is released, e.g. into the surrounding medium. Preferably, up to 100% of the compound is released, e.g. into the surrounding medium, within 8 hours, 12 hours, 24 hours, 36 hours or 48 hours, Said surrounding medium may be air, a buffered solution, a physiological buffered solution, body fluid such as blood, lymph, or liquor, or water.

The sustained or controlled release of the compound increases/prolongs the effect of the compound, e.g. pharmaceutical compound such as drug, a detergent compound such as a cleaning agent or a laundry detergent or cosmetic compound such as fragrance or fragrance oil.

In one embodiment, the aqueous formulation comprising a structural protein and an alcohol is a hydrogel comprising a structural protein and an alcohol. In this case,

the hydrogel comprising a structural protein and an alcohol according to the first or third aspect, or the article according to the fifth aspect is used for sustained or controlled release of a compound.

In one preferred embodiment, the aqueous formulation comprising a structural protein and an alcohol is a flowable hydrogel comprising a structural protein and an alcohol. In this case,

the flowable hydrogel comprising a structural protein and an alcohol according to the first or third aspect, or

the article according to the fifth aspect

is used for sustained or controlled release of a compound.

In one another preferred embodiment, the aqueous formulation comprising a structural protein and an alcohol is a non-flowable hydrogel comprising a structural protein and an alcohol. In this case,

the non-flowable hydrogel comprising a structural protein and an alcohol according to the first or third aspect, or

the article according to the fifth aspect

is used for sustained or controlled release of a compound.

In an eleventh aspect, the present invention relates to the use of

the aqueous formulation comprising a structural protein and an alcohol according the first or third aspect, or

the article according to the fifth aspect

for prolongation of the retention time of a compound.

In particular, the aqueous formulation or article comprises a compound. The compound may be selected from the group consisting of a pharmaceutical compound, a detergent compound, a cosmetic compound, a chemical compound, and a coloring compound.

The present inventors have noted that the aqueous formulation is suitable for the prolongation of the retention time of a compound.

Compared to an aqueous formulation comprising a compound and a structural protein, the retention time of a compound from an aqueous formulation comprising a compound, a structural protein, and an alcohol can be prolonged by at least 10, 15, 20, 25, 30, 35, 40, 45, 50, 60, 70, 80, 90, or 100%.

In one embodiment, the aqueous formulation comprising a structural protein and an alcohol is a hydrogel comprising a structural protein and an alcohol. In this case,

the hydrogel comprising a structural protein and an alcohol according to the first or third aspect, or

the article according to the fifth aspect

is used for the prolongation of the retention time of a compound.

In one preferred embodiment, the aqueous formulation comprising a structural protein and an alcohol is a flowable hydrogel comprising a structural protein and an alcohol. In this case,

the flowable hydrogel comprising a structural protein and an alcohol according to the first or third aspect, or

the article according to the fifth aspect

is used for the prolongation of the retention time of a compound.

In one another preferred embodiment, the aqueous formulation comprising a structural protein and an alcohol is a non-flowable hydrogel comprising a structural protein and an alcohol. In this case,

the non-flowable hydrogel comprising a structural protein and an alcohol according to the first or third aspect, or

the article according to the fifth aspect

is used for the prolongation of the retention time of a compound.

In a twelfth aspect, the present invention relates to the use of

the aqueous formulation comprising a structural protein and an alcohol according to the first or third aspect, or

the article according to the fifth aspect

for the formulation of a poorly water soluble, a water insoluble, a lipophilic, or an oily compound.

In particular, the aqueous formulation or article comprises a compound. The compound may be selected from the group consisting of a pharmaceutical compound, a detergent compound, a cosmetic compound, a chemical compound, and a coloring compound.

The present inventors have noted that the aqueous formulation is suitable for the formulation of a poorly water soluble, water insoluble, lipophilic, or oily compound.

In one embodiment, the aqueous formulation comprising a structural protein and an alcohol is a hydrogel comprising a structural protein and an alcohol. In this case,

the hydrogel comprising a structural protein and an alcohol according to the first or third aspect, or

the article according to the fifth aspect

is used for the formulation of a poorly water soluble, a water insoluble, a lipophilic, or an oily compound.

In one preferred embodiment, the aqueous formulation comprising a structural protein and an alcohol is a flowable hydrogel comprising a structural protein and an alcohol. In this case,

the flowable hydrogel comprising a structural protein and an alcohol according to the first or third aspect, or

the article according to the fifth aspect

is used for the formulation of a poorly water soluble, a water insoluble, a lipophilic, or an oily compound.

In one another preferred embodiment, the aqueous formulation comprising a structural protein and an alcohol is a non-flowable hydrogel comprising a structural protein and an alcohol. In this case,

the non-flowable hydrogel comprising a structural protein and an alcohol according to the first or third aspect, or

the article according to the fifth aspect

is used for the formulation of a poorly water soluble, a water insoluble, a lipophilic, or an oily compound.

Various modifications and variations of the invention will be apparent to those skilled in the art without departing from the scope of invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art in the relevant fields are intended to be covered by the present invention.

### BRIEF DESCRIPTION OF THE FIGURES

The following figures and examples are merely illustrative of the present invention and should not be construed to limit the scope of the invention as indicated by the appended claims in any way.

FIG. 1: Shows from left to right the mean values G′ (Pa) at γ1% (LVE) versus different protein contents (%) of C₈, C₁₆, C₃₂ and C₄₈ silk hydrogels. The samples have been determined in triplicate. (C₈: protein concentration from 1.5% (w:w) to 1.75% (w:w), C₁₆: protein concentration from 0.5% (w:w) to 1.5% (w:w), C₃₂: protein concentration from 0.5% (w:w) to 1.25% (w:w), C₄₈: protein concentration from 0.25% (w:w) to 1.17% (w:w)). It can be shown, that an increase of protein concentration result in an increase of the complex viscosity of the protein and an increase of the molecular weight of the protein result in an increase of the complex viscosity of the protein.

FIG. 2: Shows the sent intensity determined by 26 test persons of the fragrance Phenetylethanol released by compositions with 0.25% structural C₁₆ protein (SSP), compositions comprising 0.25% Dipropylenglycol (Dipro), 0.25% Tegosoft M (Tego) or negative control (Neg.) 10 min, 20 min, 30 min, 40 min, 60 min and 80 min after application of the fragrance to a test strip. The sent intensity released by the composition with structural protein (SSP) is significantly higher than the sent intensity of the fragrance released by the composition comprising Dipropylenglycol (Dipro), Tegosoft M (Tego) or negative control (Neg.). The higher release of fragrance after 10 min by the composition with structural protein (SSP) compared to the release by the compositions with Dipropylenglycol (Dipro), Tegosoft M (Tego) or the negative control (Neg.) reflects the sustained release of the compound.

FIG. 3: Shows the three options of producing an aqueous formulation comprising a structural protein and an alcohol described in the present invention. Option 1: an aqueous solution comprising a structural protein and an aqueous solution comprising an alcohol are provided and the aqueous solution comprising an alcohol is added to the aqueous solution comprising a structural protein. Option 2: an aqueous solution comprising a structural protein and an aqueous solution comprising an alcohol are provided and the aqueous solution comprising an alcohol and the aqueous solution comprising a structural protein are simultaneously brought together/combined. Option 3: an aqueous solution comprising a structural protein and an aqueous solution comprising an alcohol are provided and the aqueous solution comprising an alcohol is undercoated/underlayered by the aqueous solution comprising a structural protein.

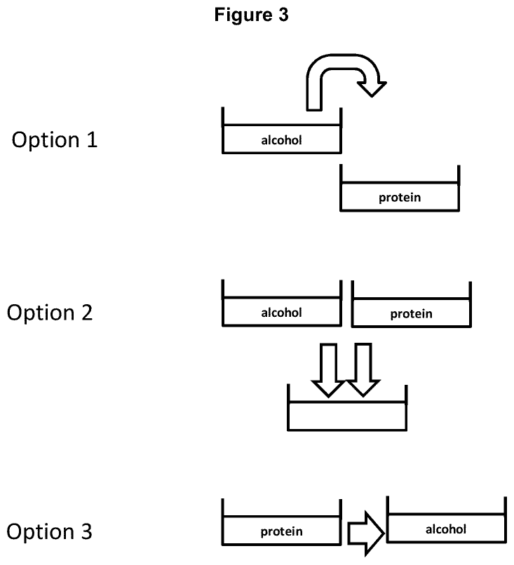

### EXAMPLES

The examples given below are for illustrative purposes only and do not limit the invention described above in any way.

### Example 1

### Preparation of C₈, C₁₆, C₃₂ and C₄₈ Silk Hydrogels

a) Preparation of C₈, C₁₆, C₃₂ and C₄₈ protein:

The C₁₆ protein (SEQ ID NO: 3) was prepared as described in WO 2006/008163. C₈ (SEQ ID NO: 6, C₃₂ protein (SEQ ID NO: 4) and C₄₈ (SEQ ID NO: 5) protein have been prepared analogous to the same process.

b) Preparation of an aqueous C₈, C₁₆, C₃₂ and C₄₈ protein solution:

For the preparation of the protein solutions, the silk proteins were dissolved in 6 M GdmSCN and 50 mM Tris/HCl, pH 8.0. In order to remove the GmdSCN, the protein solution was either dialyzed against 5 mM Tris/HCl, pH 8.0 using a Spectra/Por Dialysis Membrane with a MWCO of 6000-8000. After dialysis, the protein solution was filtered via crossflow filtration (VIVAFLOW 200, Hydrosat, 10 kDa) in order to further remove the GmdSCN and to concentration the protein in the solution.

When the volume of the protein solution was >500 mL, the GmdSCN can be removed and the protein solution concentrated without dialysis using a crossflow unit (Sartorius AG, Göttingen) with SARTOCON Slice Cassettes (Filter material: Hydrosat with 10 kDa cut off). The C₈, C₁₆, C₃₂ and C₄₈ protein concentrations were determined by measuring the absorbance at 276 nm using the UV/Vis spectroscopy (Beckman Coulter). The final protein concentrations of the C₈, C₁₆, C₃₂ and C₄₈ protein solution were between 3.75% and 6.65% (w/w).

c) Preparation of C₈, C₁₆, C₃₂ and C₄₈ silk hydrogels in 70% EtOH (Option 1):

For the preparation of silk hydrogels with a final ethanol concentration of 70%, deinonized water and 99.5% EtOH were mixed to obtain an aqueous solution with the respective EtOH concentration. This aqueous EtOH solution was added to a first beaker glass. Aqueous protein solutions (C₈, C₁₆, C₃₂ and C₄₈), prepared as described above, were added to a second beaker glass. The aqueous EtOH solution (first beaker glass) was added in one motion/at once to the aqueous protein solution in the second beaker glass and promptly mixed by agitating and subsequently slewing the mixture. The addition of the aqueous EtOH/deinonized water solution had to be carried out within no more than 5 seconds.

The final concentrations of C₈ silk hydrogels were 1.35% (w/w), 1.5% (w/w), 1.625% (w/w) and 1.75% (w/w) in a final concentration of 70% EtOH. Silk hydrogels with a protein concentration of up to 1.625% (w/w) result in a flowable hydrogel.

The final concentrations of C₁₆ silk hydrogels were 0.5% (w/w), 1.0% (w/w), 1.25% (w/w), 1.5% (w/w) and 2.0% (w/w) in a final concentration of 70% EtOH. Silk hydrogels with a protein concentration up to 1.25% (w/w) result in a flowable hydrogel. Silk hydrogels with protein concentrations of 1.5% (w/w) and 2.0% (w/w) result in a non-flowable hydrogel.

The final concentrations of C₃₂ silk hydrogels were 0.5% (w/w), 0.75% (w/w), 1.0% (w/w) and 1.25% (w/w) in a final concentration of 70% EtOH. Silk hydrogels with a protein concentration up to 0.75 (w/w) result in a flowable hydrogel. Silk hydrogels with protein concentrations of 1.0% (w/w) and 1.25% (w/w) result in a non-flowable hydrogel.

The final concentrations of C₄₈ silk hydrogels were 0.25% (w/w), 0.5% (w/w), 0.75% (w/w), 1.0% (w/w) and 1.165% (w/w) in a final concentration of 70% EtOH. Silk hydrogels with a protein concentration up to 0.5% (w/w) result in a flowable hydrogel. Silk hydrogels with protein concentrations of 0.75% (w/w), 1.0% (w/w) and 1.165% (w/w) result in a non-flowable hydrogel.

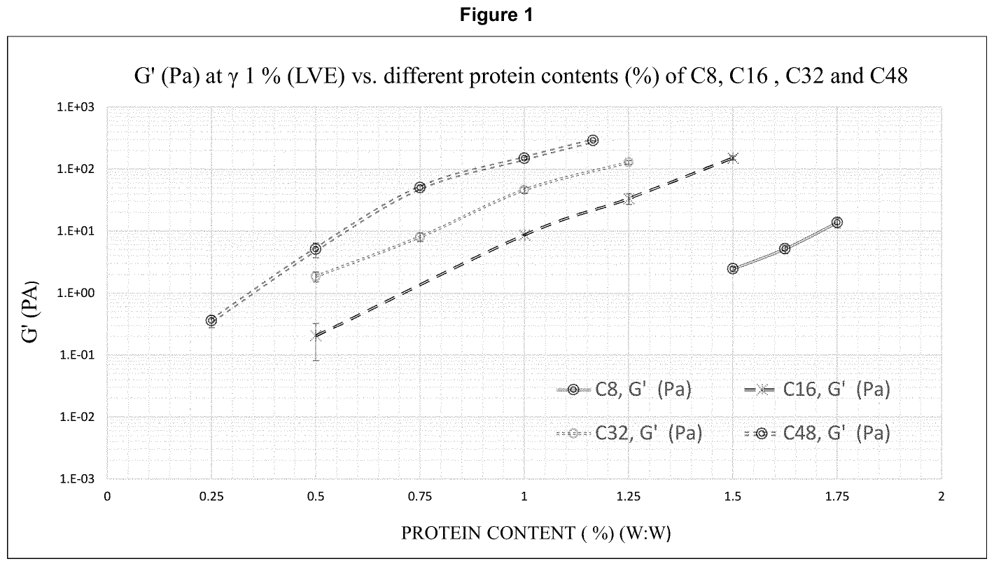

The complex viscosities of the hydrogels are shown in FIG. 1.

An increase of the molecular weight of the protein result in an increase of viscosity.

The examples show that lower protein concentrations result in a non-flowable hydrogel the higher the molecular weight of the protein is. A person skilled in the art can determine the respective concentration in order to obtain a flowable or a non-flowable hydrogel.

### Example 2

### Determination of the Complex Viscosity of the Silk Hydrogels:

The complex viscosities of the silk hydrogels produced in Example 1 have been determined in a cone-plate measuring system (Modular Compact Rheometer Manufacturer: Anton Paar Type: MCR 102, Measurement cone: CP25-1, d: 25 mm, angle: 1° (Serial No.: 31081) according to the manufactures manual with the following parameters:

Value: γ Shear deformation (oscillating)

Profile: ramp logarithmic

Start value: 0.01%

End value: 100%

Value: ω (rad/s) circle-frequency

Profile: constant

Value: 10 rad/s

Sample measurement temperature (Plate): 15° C.

Measurement gap: 50 μm

Evaluate parameter for description the silk gel viscosity: shear deformation at γ1% (LVE)->G′ (Pa).

The complex viscosities of the C₈, C₁₆, C₃₂ and C₄₈ silk hydrogels have been determined in triplicate. The mean values G′ (Pa) at γ1% (LVE) versus different protein contents (%) of C₈, C₁₆, C₃₂ and C₄₈ silk hydrogels are shown in FIG. 1. It can be shown, that an increase of protein concentration result in an increase of the complex viscosity of the protein and an increase of the molecular weight of the protein result in an increase of the complex viscosity of the protein.

C₁₆ protein correspond to molecular weight of 47.7 kDa. C₃₂ protein correspond to molecular weight of 93.8 kDa and C₄₈ protein correspond to a molecular weight of 139.9 kDa. The higher the complex viscosity of the protein the lower protein concentration result in a non-flowable hydrogel. The lower the complex viscosity of the protein the higher protein concentration result in a non-flowable hydrogel. This means that higher concentrations of proteins with lower molecular weight can be formed into a flowable hydrogel than with higher molecular weight proteins respectively that flowable hydrogels with higher concentrations can be achieved with proteins of lower molecular weight/lower complex viscosity than with proteins of higher molecular weight/higher complex viscosity.

A person skilled in the art can determine the respective concentrations of a protein needed in order to obtain a flowable or non-flowable hydrogel considering the molecular weight respectively the complex viscosity of the protein. Alternatively the respective concentrations of a protein needed in order to obtain a flowable or non-flowable hydrogel can be determined empirically for example by a dilution series of the respective protein concentration. In order to determine the complex viscosity of a protein the sequence of the amino acids of the protein has to be considered as well as the content of hydrophilic or hydrophobic amounts in the protein.

### Example 3

### Alternative Preparation of C₁₆, Silk Hydrogels in 70% EtOH

a) Preparation of a C₁₆ Silk hydrogel with a protein concentration of 0.75% (w/w) and 1.5% in 70% EtOH via simultaneous mixing in a mixing chamber (Option 2):

The C₁₆ protein (SEQ ID NO: 3) and the aqueous C₁₆ protein solution were prepared as described in Example 1. An aqueous EtOH solution (99.5% EtOH) was added to a first reaction vessel and an aqueous C₁₆ protein solution with 3.3% or 6.6% (w:w) protein respectively was added to a second reaction vessel. Both solutions were simultaneously combined in a mixing chamber and mixed with a magnetic stirrer so that a hydrogel with a protein concentration of 0.75% (w/w) or 1.5% (w/w) was formed. The reaction vessels were connected with the mixing chamber by flexible tubes. The aqueous EtOH solution was fed to the aqueous protein solution in the mixing chamber in a mixing ratio of 4.3:1 (EtOH solution:protein solution).

Silk hydrogels with a protein concentration of 0.75% (w/w) result in a flowable hydrogel.

Silk hydrogels with a protein concentration of 1.5% (w/w) result in a non-flowable hydrogel.

b) Preparation of a C₁₆ Silk hydrogel with a protein concentration of 0.75 (w/w) and 1.5% in 70% EtOH via two-phase liquid system (Option 3):

The C₁₆ protein (SEQ ID NO: 3) and the aqueous C₁₆ protein solution were prepared as described in Example 1. In order to obtain a two-phase liquid system, an aqueous EtOH solution (99.5% EtOH) was added to a reaction tube with a stirrer and then gently underlaid with an aqueous C₁₆ protein solution with 3.3% or 6.6% (w:w) protein respectively. The resulting two-phase liquid system consisting of an aqueous EtOH phase and an aqueous protein phase was mixed with the stirrer so that a silk hydrogel with a protein concentration of 0.75% (w/w) or 1.5% (w/w) was formed.

Silk hydrogels with a protein concentration of 0.75% (w/w) result in a flowable hydrogel.

Silk hydrogels with a protein concentration of 1.5% (w/w) result in a non-flowable hydrogel.

### Example 4

### Sustained Release of a Compound from a Composition Comprising an Aqueous Formulation of a Structural Protein and an Alcohol

In order to show the sustained release of a compound, a fragrance (Phenetylethanol) as exemplary poorly water soluble compound was added to an aqueous composition comprising a structural protein and an alcohol. The sustained release of the fragrance was compared to aqueous solutions without structural protein and aqueous solutions comprising the fixative Dipropylenglycol (Carl Roth, Karlsruhe, Germany) or Tegosoft M (Franken Chemie, Wendelstein Germany). Therefore 5% Phenetylethanol (Carl Roth, Karlsruhe, Germany) was added to aqueous solutions with C₁₆ protein resulting in a concentration of 0.25% C₁₆ protein (SSP), 70% EtOH or to aqueous solutions with Dipropylenglycol resulting in a concentration of 0.25% Dipropylenglycol, 70% EtOH (Dipro), to aqueous solutions with Tegosoft M resulting in a concentration of 0.25% Tegosoft M, 70% EtOH (Tego). An aqueous solution with 70% EtOH without structural protein or fixative served as a negative control (Neg.). 100 μl of each composition containing the structural C₁₆ protein (SSP), Dipropylenglycol (Dipro), Tegosoft M (Tego) and the negative control (Neg.) were applied to a teststrip (Rotilabo®-Riechstreifen, Carl Roth, Karlsruhe, Germany).

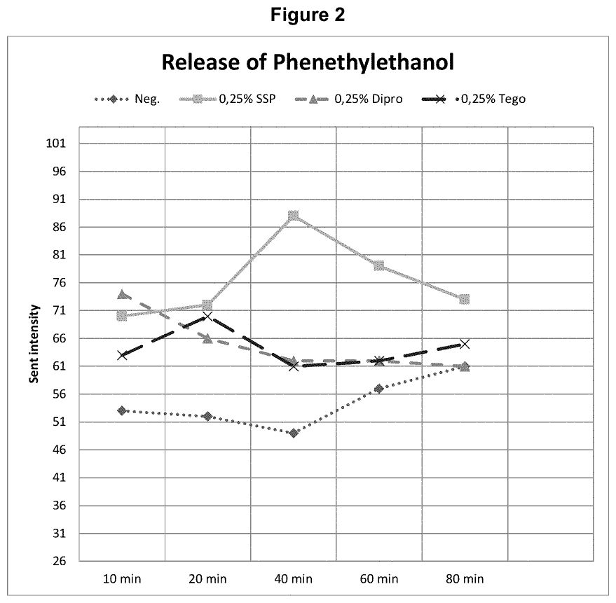

26 test persons determined the release of the fragrance by estimating the sent intensity of the fragrance 10 min, 20 min, 30 min, 40 min, 60 min and 80 min after application of the fragrance to the test strip. The sent represents the top note of a perfume which is a highly volatile scent quickly released by the medium. The sent intensity of the released fragrance Phenetylethanol in relation to the release time of the fragrance is shown in FIG. 2. It can be shown that the sent intensity of the fragrance released by the composition with structural protein (SSP) is significantly higher than the sent intensity of the fragrance released by the composition comprising Dipropylenglycol (Dipro), Tegosoft M (Tego) or negative control (Neg.). The higher release of fragrance released after 10 min by the composition with structural protein (SSP) compared to the release by the compositions with Dipropylenglycol (Dipro), Tegosoft M (Tego) or the negative control (Neg.) reflects the sustained release of the compound.

The use of the inventive protein-alcohol solution allows the sustained release of fragrances without the help of fixatives. In addition less amount of fragrance is needed to obtain a sustained and long lasting release profile for fragrances.

1. An aqueous formulation comprising a structural protein and an alcohol.

2. The aqueous formulation of claim 1, wherein said formulation has a clear appearance.

3. The aqueous formulation of claim 1 wherein the aqueous formulation comprises
between 60 wt % and 90 wt % alcohol,
between 0.05 wt % and 5 wt % structural protein, and
between 5 wt % and 39.95 wt % water.

4. The aqueous formulation of claim 1, wherein the alcohol is selected from the group consisting of: ethanol, methanol, and isopropanol.

5. The aqueous formulation of claim 1, wherein the structural protein has a molecular weight of between 20 kDa and 140 kDa.

6. The aqueous formulation of claim 1, wherein the aqueous formulation has a complex viscosity of between 0.04 Pa·s and 30 Pa·s.

7. The aqueous formulation of claim 1, wherein the aqueous formulation is a hydrogel.

8. The aqueous formulation of claim 1, wherein the structural protein is a self-assembling protein.

9. The aqueous formulation of claim 1, wherein the structural protein is selected from the group consisting of: a silk protein, keratin, collagen, and elastin.

10. The aqueous formulation of claim 9, wherein the silk protein is a recombinant silk protein.

11. The aqueous formulation of claim 9, wherein the silk protein comprises at least two identical repetitive units.

12. The aqueous formulation of claim 11, wherein the repetitive units are independently selected from the group consisting of: module C having the sequence according to SEQ ID NO: 1 or a variant thereof, and module C^{Cys} having the sequence according to SEQ ID NO: 2 or a variant thereof.

13. The aqueous formulation of claim 1, wherein the aqueous formulation further comprises a compound.

14. The aqueous formulation of claim 13, wherein the compound is poorly water soluble, water insoluble, lipophilic, or oily.

15. The aqueous formulation of claim 13, wherein the compound is selected from the group consisting of: a pharmaceutical compound, a detergent compound, a cosmetic compound, a chemical compound, and a coloring compound.

16. A method for producing an aqueous formulation comprising a structural protein and an alcohol comprising the steps of:
(i) providing an aqueous solution comprising a structural protein and an aqueous solution comprising an alcohol, and
(ii) mixing the aqueous solutions, thereby obtaining an aqueous formulation comprising a structural protein and an alcohol.

17. The method of claim 16, wherein the method further comprises subsequent to step (i) a step of: adding the aqueous solution comprising an alcohol to the aqueous solution comprising a structural protein.

18. The method of claim 17, wherein the aqueous solution comprising an alcohol is added to the aqueous solution comprising a structural protein
in one motion/at once, or
within no more than 10 seconds.

19. The method of claim 17, wherein the mixing is performed by avoiding the application of shear forces.

20. The method of claim 16, wherein the method further comprises subsequent to step (i) a step of: simultaneously bringing together/combining the aqueous solution comprising a structural protein and the aqueous solution comprising an alcohol.

21. The method of claim 20, wherein the aqueous solutions are mixed for no more than 10 seconds.

22. The method of claim 16, wherein the method further comprises subsequent to step (i) a step of: undercoating/underlayering the aqueous solution comprising an alcohol with the aqueous solution comprising a structural protein.

23. The method of claim 22, wherein the aqueous solutions are mixed for no more than 10 seconds.

24. The method of claim 16, wherein the concentration of the structural protein in the aqueous solution provided in (i) is of between 0.05 wt % and 5 wt %.

25. The method of claim 16, wherein the concentration of the alcohol in the aqueous solution added in step (i) is of between 50 wt % and 90 wt %.

26. The method of claim 16, wherein the aqueous solution comprising a structural protein is homogenous.

27. The method of claim 16, wherein said aqueous formulation is a hydrogel.

28. The method of claim 16, wherein the alcohol is selected from the group consisting of: ethanol, methanol, and isopropanol.

29. The method of claim 16, wherein the structural protein has a molecular weight of between 20 kDa and 140 kDa.

30. The method of claim 16, wherein the method further comprises the step of: adding a compound to:
the aqueous solution comprising a structural protein provided in step (i),
the aqueous solution comprising an alcohol provided in step (i), and/or
the mixture in step (ii).

31. The method of claim 30, wherein the compound is poorly water soluble, water insoluble, lipophilic, or oily.

32. The method of claim 30, wherein the compound is selected from the group consisting of: a pharmaceutical compound, a detergent compound, a cosmetic compound, a chemical compound, and a coloring compound.

33. The method of claim 16, wherein the structural protein is a self-assembling protein.

34. The method of claim 16, wherein the structural protein is selected from the group consisting of: a silk protein, keratin, collagen, and elastin.

35. The method of claim 34, wherein the silk protein is a recombinant silk protein.

36. The method of claim 34, wherein the silk protein comprises at least two identical repetitive units.

37. The method of claim 36, wherein the repetitive units are independently selected from the group consisting of: module C having the sequence according to SEQ ID NO: 1 or a variant thereof, and module C^{Cys} having the sequence according to SEQ ID NO: 2 or a variant thereof.

38. An aqueous formulation comprising a structural protein and an alcohol produced by the method of claim 16.

39. A method for producing an article comprising the steps of:
(i) providing an aqueous formulation comprising a structural protein and an alcohol according to claims claim 1, and
(ii) forming an article out of/from the said aqueous formulation.

40. The method of claim 39, wherein the method further comprises the step of: adding a compound to the aqueous formulation provided in step (i) or to the article formed in step (ii).

41. The method of claim 40, wherein the compound is poorly water soluble, water insoluble, lipophilic, or oily.

42. The method of claim 40, wherein the compound is selected from the group consisting of: a pharmaceutical compound, a detergent compound, a cosmetic compound, a chemical compound, and a coloring compound.

43. An article produced by the method of claim 39.

44. A pharmaceutical composition comprising:
the aqueous formulation comprising a structural protein and an alcohol according to claim 1.

45. A cosmetic composition comprising:
the aqueous formulation comprising a structural protein and an alcohol according to claim 1.

46. (canceled)

47. A method of protecting a compound, the method comprising the step of:
utilizing
the aqueous formulation comprising a structural protein and an alcohol according to claim 1
to protect the compound.

48. The method of claim 47, wherein the compound is selected from the group consisting of: a pharmaceutical compound, a detergent compound, a cosmetic compound, a chemical compound, and a coloring compound.

49. A method of providing sustained or controlled release of a compound, the method comprising the step of:
utilizing the aqueous formulation comprising a structural protein and an alcohol according to claim 1
to provide sustained or controlled release of the compound.

50. The method of claim 49, wherein the compound is selected from the group consisting of: pharmaceutical compound, a detergent compound, a cosmetic compound, a chemical compound, and a coloring compound.

51. A method of prolonging the retention time of a compound, the method comprising the step of:
utilizing
the aqueous formulation comprising a structural protein and an alcohol according to claim 1
to provide prolongation of the retention time of the compound.

52. The method of claim 51, wherein the compound is selected from the group consisting of: a pharmaceutical compound, a detergent compound, a cosmetic compound, a chemical compound, and a coloring compound.

53. A method of formulating a poorly water soluble, a water insoluble, a lipophilic, or an oily compound, the method comprising the step of: Use of utilizing the aqueous formulation comprising a structural protein and an alcohol according to claim 1
in the formulation of the poorly water soluble, a water insoluble, a lipophilic, or an oily compound.

54. The method claim 53, wherein the compound is selected from the group consisting of: a pharmaceutical compound, a detergent compound, a cosmetic compound, a chemical compound, and a coloring compound.
